(19) United States
(12) Patent Application Publication
(10) Pub. No.: US 2024/0105289 A1
(43) Pub. Date: Mar. 28, 2024

(54) CLINICAL ENDPOINT ADJUDICATION SYSTEM AND METHOD

(71) Applicant: ASTRAZENECA AB, SODERTALJE (SE)

(72) Inventors: FAISAL KHAN, WILMINGTON, DE (US); ELISABETH BJORK, SODERTALJE (SE); TOMAS ANDERSON, SODERTALJE (SE); ANDERS PERSSON, SODERTALJE (SE); CRISTINA DURAN, CAMBRIDGE (GB); GLYNN DENNIS, WILMINGTON, DE (US); SHAMEER KHADER, WILMINGTON, DE (US); EMMETTE HUTCHISON, WILMINGTON, DE (US); HALSEY LEA, WILMINGTON, DE (US); SREENATH NAMPALLY, WILMINGTON, DE (US); MALIN WALLANDER, SODERTALJE (SE); ANDREAS JAREMO, SODERTALJE (SE)

(21) Appl. No.: 18/262,934

(22) PCT Filed: Jan. 25, 2022
(86) PCT No.: PCT/EP2022/051564

§ 371 Date: Jul. 26, 2023

Related U.S. Application Data
(60) Provisional application No. 63/141,554, filed on Jan. 26, 2021.

(51) Int. Cl. G16H 10/20; G06F 40/40; G16H 50/20; G16H 50/70

(52) U.S. Cl. CPC G16H 10/20; G06F 40/40; G16H 50/20; G16H 50/70

(57) ABSTRACT

The present disclosure relates to a system and method for use in performing clinical endpoint adjudication to provide: efficient, automated clinical event classification and medical review triage; reduce the time to identify clinical events; a unified, consistent process for classification of clinical events; and proactive identification of events in near real-time.

### FIELD OF THE INVENTION

The present disclosure relates to a system and method for use in performing clinical endpoint adjudication.

### BACKGROUND

Outcome trials are a regulatory requirement for cardiovascular, renal and metabolism (CVRM) projects to demonstrate safety and efficacy/benefit. They require a large sample size (several thousands of patients) and are expensive to run. Clinical endpoint event adjudication is the process by which an independent, blinded expert committee reviews clinical events that occur during the trial. These are assessed—adjudicated—against a set of pre-defined criteria to classify the events. This is because otherwise a local investigator or doctor may have their own opinion as to what type of event the patient had, which could result in a high degree of variability. By using a blinded expert committee this variability can be reduced. Endpoint adjudication can be used to assess both efficacy outcomes as well as safety outcomes and has been used for 25-30 years across the industry and by all major pharma companies. It provides an independent review of the event, avoiding bias from regional differences and individual investigators.

However, event adjudication represents 5% of the average CVRM outcome trial cost. It is a manual and iterative process. It requires highly skilled clinicians that are occupied by easy-to-evaluate events. It generates duplication of adjudication data across multiple systems. It is therefore time consuming and costly for the sponsor and can delay the drug development life cycle.

Clinical endpoint event adjudication is expensive and resource intensive (roughly $8.5 M per large CVRM outcome study). It can result in approximately 4-5 months delay which includes event capture and manual adjudication process. It is reliant on secondary or tertiary reporting and is a largely manual and iterative process. Capturing all events is essential, as missed events can influence the outcome of a trial. When large, multi-centre and multi-country studies are performed, this problem is amplified.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

In a first aspect of the disclosure there is provided a computer-implemented method for performing clinical trial endpoint adjudication. The method comprises, at computer system or device, receiving data from a plurality of healthcare-related data sources. Optionally, the method comprises analysing each data source to determine whether data held by the data source comprises structured and/or unstructured data. In the event that the data comprises unstructured data, the method comprises applying a natural language processing model to the unstructured data to obtain embeddings relating to features in the unstructured data. In the event that the data comprises structured data, the method comprises extracting features from that data. The method further comprises applying a machine learning classification model to the embeddings from the unstructured data and the features extracted from the structured data to classify whether a healthcare event has occurred based on the embeddings and the features extracted from the structured data.

Optionally, the method further comprises attributing a probability score as an attribute to the classification, wherein the probability score provides an indication of the likelihood of the event having occurred; and providing a notification to a user to review classifications where the probability score is less than a selected threshold.

Applying a natural language processing model may comprise applying a plurality of natural language processing models, for example comprising a first specialised model trained on text available from the data sources, with a second general model trained on, for example, Wikipedia®.

In the event that the data comprises unstructured data, the method may comprise applying a named-entity recognition model to the unstructured data to obtain formal event characteristics from the unstructured data, and applying the machine learning classification model to the formal event characteristics obtained via the named-entity recognition model.

The method may further comprise attributing a confidence score as an attribute to the data based on at least one of (i) the data source, and (ii) a determined confidence based on an optical character recognition process applied to the unstructured data, and using the confidence score as a weighting by the machine learning classification model. For example, data acquired from a known or trusted source may be given a higher weighting than data acquired from an unknown or unreliable source. In some examples, only data with a weighting above a selected threshold may be used—for example so that erroneous or unreliable data is not used that could distort the method. Consequently, the method may comprise excluding data having a confidence score below a selected threshold.

In some examples extracting features from the data and applying a natural language processing model to the unstructured data to obtain embeddings relating to features in the unstructured data comprises obtaining a predefined set of features for use in the clinical endpoint adjudication and mapping the extracted features and/or embeddings to the predefined set of features, and discarding features and/or embeddings that do not relate to the predefined set of features.

The machine learning classification model may provide a ranking of the importance of the features involved in classifying whether a healthcare event has occurred. This may be helpful for regulatory purposes and/or for diagnostic purposes to show how the model is performing and on what features decisions are being made. Providing a ranking of the importance of features may comprise determining the SHAP value for each feature. Additionally, or alternatively, providing a ranking of the importance of features may comprise applying a local surrogate model to the machine learning classification model to determine the relative contribution of each feature to the classification.

The method may be performed when the amount of data available exceeds a selected threshold. In this way, classification may only be performed when enough data is available to perform an endpoint adjudication decision. Additionally, or alternatively, the method may be performed in response to an indication of an event having occurred being provided by a user.

In another aspect there is provided a method of training a machine learning classification model for performing clinical trial endpoint adjudication. The method comprises receiving data from a plurality of healthcare-related data sources, the data comprising adjudication dossiers from previous clinical trials and adjudication decisions relating to those adjudication dossiers, and analysing each data source to determine whether data held by the data source comprises structured and/or unstructured data. In the event that the data comprises unstructured data, applying a natural language processing model to the unstructured data to obtain embeddings relating to features in the unstructured data. In the event that the data comprises structured data, extracting features from that data. The method further comprises providing an indication of the adjudication decision based on the data from the adjudication dossier, updating the machine learning classification model based on the adjudication decision and the data from the adjudication dossier, and storing the updated machine learning classification model in a relational database.

In another aspect there is provided a method of monitoring clinical trial endpoint adjudication. The method comprises receiving a plurality of notifications of an adjudication decision from a clinical trial endpoint adjudication system, wherein the adjudication decision comprises a probability score providing an indication of the likelihood of the event having occurred, ranking the notifications based on at least one of (i) the probability score and (ii) the severity of the event, obtaining a dossier of data used in performing the adjudication, and providing a list of adjudication decisions and the corresponding dossier of data to a user to review the correctness of the adjudication decision, wherein the order of the list is based on the ranking. Such a method may help to ensure that the clinical trial endpoint adjudication process is kept efficient and to help ensure that when input is needed, for example, from a healthcare professional, that this is obtained in a timely manner.

The method may further comprise obtaining a ranking of the importance of the features involved in classifying whether a healthcare event has occurred, and providing the ranking of the importance of the features to the user with the list of adjudication decisions and the corresponding dossier of data. They may help to identify whether any input required is overdue and/or if, for example, input is needed urgently from a healthcare professional.

In another aspect there is provided a computer-implemented method of harmonising and collating data from a plurality of healthcare-related sources for clinical trial endpoint adjudication. The method comprises analysing each data source to determine whether data held by the data source comprises structured and/or unstructured data. In the event that the data comprises unstructured data, the method comprises performing optical character recognition on a region or regions of the data not already in a machine-readable format. The method further comprises attributing a confidence score as an attribute to the data based on at least one of (i) the data source, and (ii) a determined confidence based on the optical character recognition process, performing a feature analysis on the data to extract features from the data, mapping the extracted features to a predefined set of features, and publishing the mapped extracted features in a json format for use by a machine learning model in performing the clinical trial endpoint adjudication, wherein the confidence score is an attribute of the feature.

In some examples the method comprises publishing the mapped extracted features in, for example, a json format, when the confidence score is above a selected confidence threshold.

The method may further comprise obtaining a set of features needed for a clinical trial endpoint adjudication, wherein the set of features needed is based on the endpoint;
comparing the features obtained from the plurality of data sources with the set of features needed for a clinical trial endpoint adjudication to determine whether any features are missing or incomplete;
in the event that it is determined that any features are missing or incomplete, providing a notification to a user that features are missing, the notification providing an indication of the missing or incomplete features.

It will be understood that depending on the clinical endpoint being considered (e.g. death vs myocardial infarction, for example), different features will be needed for adjudication.

In some examples the method may further comprise determining the set of features needed for the clinical trial endpoint adjudication.

The method may further comprise performing named-entity recognition on the data prior to performing a feature analysis on the data and selecting formal event characteristics that are relevant to the predefined set of features.

In some examples, the method comprises obtaining a set of features that should be (or are expected to be) provided by a data source, and determining whether any features are missing for that data source (for example by making a comparison with a set of expected features), and in the event that features are missing for that data source, providing a notification to a user that features are missing.

In some examples performing a feature analysis on the data to extract features from the data further comprises checking for and removing any duplicated, inconsistent or inapplicable features.

In another aspect there is provided a monitoring system for determining whether a healthcare event has occurred for a participant in a clinical trial. The system comprises a communications interface configured to receive data signals relating to a plurality of participants from a plurality of sources, wherein the data signals each comprise information indicative of a parameter associated with a participant, and a processor. For each participant, the processor is configured to process each received data signal and apply a first weighting to each data signal based on the source of the data signal. It will be understood that this weighting could be based on business rules—for example, this type of signal could be defined as an important “trigger” signal, this type of signal could be defined as a “contextual” signal that provides useful information that may help in determining whether an event has occurred but cannot be used on its own for such a determination. The processor is configured to make a determination of the probability of a healthcare event occurring based on at least one of: (i) the data signal indicating that a parameter associated with a patient exceeds a selected threshold for that participant (for example, a patient is within a selected range of a hospital for greater than a selected time duration), and (ii) the first weighting exceeding a selected trigger threshold (for example, the signal is a “trigger” signal rather than a “contextual” signal, and/or a sufficient number of “contextual” signals have been obtained to allow a determination to be made).

The processor may be configured to make a determination that a healthcare event has occurred based on the determined probability exceeding a selected threshold, and in the event that the processor determines that a healthcare event has occurred, the processor is configured to provide a notification to a user of the monitoring system, and wherein the monitoring system is configured to rank the notifications based on the determined probability of the healthcare event occurring.

The processor may be configured to make a determination of the type of healthcare event based on the source of the data signal and the indication of the parameter associated with the participant.

In some examples the monitoring system is configured to rank the notifications based on the determined type of healthcare event—for example, more severe events (for example, as held in a look up table, for example), may be ranked more highly. Additionally, or alternatively, the monitoring system may be configured to rank the notifications based on the known health of the participants.

The processor may be configured to make a determination of the probability of a healthcare event occurring based on a plurality of data signals with at least one data signal indicating at least one of (i) that a parameter associated with a patient exceeds a selected threshold and that (ii) a weighting of that data signal exceeds a selected threshold. For example, an algorithm or multiplication function may be used to combine the data signals in a particular way.

In some examples the processor makes a determination of the probability of a healthcare event occurring based on a received data signal indicating that a parameter has exceeded a selected threshold, wherein the processor reviews information indicative of previous values of that parameter associated with the participant in a selected time interval preceding the determination.

A series of internal rules may define these selected time intervals—for example whether and how long for previous events/data is looked through. For example, the process may be configured to review e.g. blood pressure over course of week and e.g. heart rate over course of previous 12 hours. These windows could also vary on the device being used (e.g. its reliability). In some examples the processor may be configured to review historic data only in the event that a parameter exceeds a selected threshold.

In some examples, the processor may be configured to obtain multiple, repeated measurements to give a degree of verifiability. The processor may be configured to distinguish between measurement error (e.g. missing data/poor data quality) and potential safety event (e.g. single point anomaly of elevated respiratory rate, or trend anomaly of weight gain).

In some examples, if it is determined that the probability of an event having occurred exceeds a selected threshold, then the processor is configured to make a determination as to whether more information is needed from the patient, and in the event that more information is needed, then a notification is provided to the health care provider/system administrator to contact the patient. For example, the processor may be configured to compare the obtained information against a look up table of information that is known to be needed, and/or to previous determinations made, to determine whether it has enough information.

The processor may also be configured to make a determination of the reliability of the data signal, and to apply a second weighting based on the reliability (e.g. not uploaded, not performed correctly, poor connection, low battery) of the data signal, and wherein the processor is configured to make a determination of the probability of a healthcare event occurring based on the data signal indicating that a parameter associated with a patient exceeds a selected threshold and the first and second weightings.

The processor may be configured to make a determination of the probability of a healthcare event occurring for a participant also based on any previous determined probabilities of an event occurring for that participant.

In another aspect there is provided a method for determining whether a healthcare event has occurred for a participant in a clinical trial. The method comprises receiving data signals relating to a plurality of participants from a plurality of sources, wherein the data signals each comprise information indicative of a parameter associated with a participant; and for each participant, processing each received data signal and applying a first weighting to each data signal based on the source of the data signal. The method further comprises determining the probability of a healthcare event occurring based on at least one of: (i) the data signal indicating that a parameter associated with a patient exceeds a selected threshold for that participant, and (ii) the first weighting exceeding a selected trigger threshold.

The method may further comprise determining that a healthcare event has occurred based on the determined probability exceeding a selected threshold, and in the event that it is determined that a healthcare event has occurred, providing a notification to a user, wherein the notification is ranked based on the determined probability of the healthcare event occurring.

The method may further comprise determining the type of healthcare event based on the source of the data signal and the indication of the parameter associated with the participant. The method may further comprise ranking the notifications based on the determined type of healthcare event. The method may further comprise ranking the notifications based on the known health of the participants.

The method may further comprise making a determination of the probability of a healthcare event occurring based on a plurality of data signals with at least one data signal indicating at least one of (i) that a parameter associated with a patient exceeds a selected threshold and that (ii) a weighting of that data signal exceeds a selected threshold.

The method may further comprise making a determination of the probability of a healthcare event occurring based on a received data signal indicating that a parameter has exceeded a selected threshold, wherein the determination is based on information indicative of previous values of that parameter associated with the participant in a selected time interval preceding the determination.

In some examples, if it is determined that the probability of an event having occurred exceeds a selected threshold, then the processor is configured to make a determination as to whether more information is needed from the patient, and in the event that more information is needed, then a notification is provided to the health care provider/system administrator to contact the patient.

The method may further comprise making a determination of the reliability of the data signal, and applying a second weighting based on the reliability of the data signal, and wherein the method comprises making a determination of the probability of a healthcare event occurring based on the data signal indicating that a parameter associated with a patient exceeds a selected threshold and the first and second weightings.

The method may further comprise making a determination of the probability of a healthcare event occurring for a participant also based on any previous determined probabilities of an event occurring for that participant.

In another aspect there is provided a monitoring system for determining whether a healthcare event has occurred for a participant in a clinical trial. The system comprises a communications interface configured to receive data signals relating to a plurality of participants from a plurality of sources, wherein the data signals each comprise information indicative of a location associated with a participant, and a processor. For each participant, the processor is configured to process each received data signal; and the processor is configured to make a determination of the probability of a healthcare event occurring for a participant based on (i) the proximity of the participant to a known healthcare centre and (ii) the duration of the participant's proximity to the known healthcare centre. In the event that the processor determines that the probability of a healthcare event occurring exceeds a selected threshold, the processor is configured to send a notification to the participant requesting confirmation from the participant that a healthcare event has occurred.

In another aspect there is provided method for determining whether a healthcare event has occurred for a participant in a clinical trial. The method comprises receiving data signals relating to a plurality of participants from a plurality of sources, wherein the data signals each comprise information indicative of a location associated with a participant; and processing, for each participant, each received data signal to make a determination of the probability of a healthcare event occurring for a participant based on (i) the proximity of the participant to a known healthcare centre and (ii) the duration of the participant's proximity to the known healthcare centre. In the event that it is determined that the probability of a healthcare event occurring exceeds a selected threshold, the method comprises sending a notification to the participant requesting confirmation from the participant that a healthcare event has occurred.

In another aspect there is provided a computer readable non-transitory storage medium comprising a program for a computer configured to cause a processor to perform the method of any of any of the aspects described above.

### DRAWINGS

Embodiments of the disclosure will now be described, by way of example only, with reference to the accompanying drawings, in which:

FIG. 1 shows an example schematic process flow-chart of an example a conventional approach to clinical endpoint adjudication;

FIG. 2 shows an example schematic process flow-chart of an example approach to clinical endpoint adjudication according to embodiments of the disclosure;

FIG. 3 shows an example functional schematic of example of an event sniffer or monitoring system;

FIG. 4 shows an example series of screenshots of an application running on a mobile device for providing a notification to a user and for obtaining input from a user, for example who may be determined to have undergone a healthcare event;

FIG. 5 shows an example schematic process flow-chart of the functionality of an event sniffer module;

FIG. 6 shows an example process flow-chart of the steps the processor of the event sniffer or monitoring system illustrated in FIG. 3 may perform to determine whether a potential anomaly has occurred;

FIG. 7 shows an example process flow chart as to how a determination of whether an anomaly in obtaining healthcare data is made;

FIG. 8 shows the rules that are used in the example process of FIG. 7 to determine whether a healthcare event has occurred or not;

FIG. 9 shows an example process flow chart of a method of determining whether an anomaly or a healthcare event has occurred;

FIG. 10 shows another example process flow chart of a method of determining whether an anomaly or a healthcare event has occurred;

FIG. 11 shows an example functional schematic of the modules that may comprise a data harmonization and collection system;

FIG. 12 shows an example process flow chart for how data from unstructured data sources may be imported;

FIG. 13 shows the number of fields used in each of the THEM IS, DAPA-HF and DELIVER clinical trials, and the percentage overlap in the fields for the three trials;

FIG. 14 shows an example process flow chart for a user to manually review documents that have been determined to have regions with low confidence;

FIG. 15 shows an example high-level process flow chart of the steps involved when deploying the automated event adjudication module;

FIG. 16 shows an example process flow-chart of an example computer-implemented method for performing clinical endpoint adjudication;

FIG. 17 shows at a high level the components (for example, for example modules that may be implemented in software and/or hardware) and flow of data between them, along with brief descriptions of the function of each component, operable for performing the example method of FIG. 16 described above and for training a machine learning model for performing the example method of FIG. 16;

FIG. 18 shows an example of how the Automated Event Adjudication module may be used in combination with the Data harmonization and collection module shown in FIG. 2;

FIGS. 19A to 9C shows the results of 3 algorithms (CV Death, Non-CV Death and Undetermined) that were trained on 817 patients from the DECLARE clinical trial;

FIGS. 20A and 20B show how the models or algorithms used in examples of the disclosure can be analysed to provide interpretability;

FIG. 21 shows an example graph showing the relative SHAP values of different features used in an example mode;

FIG. 22 shows machine learning model performance across a number of metrics (e.g., AUC-Area Under the Curve, Accuracy, Balanced Accuracy, F1, etc.) and model versions (first three columns);

FIG. 23 shows machine learning model performance across a number of metrics (e.g., AUC-Area Under the Curve, Accuracy, Balanced Accuracy, F1, etc.) performed on data from 3 clinical trials; and

FIG. 24 shows a functional schematic block diagram of a computer system suitable for implementing one or more embodiments of the present disclosure.

### SPECIFIC DESCRIPTION

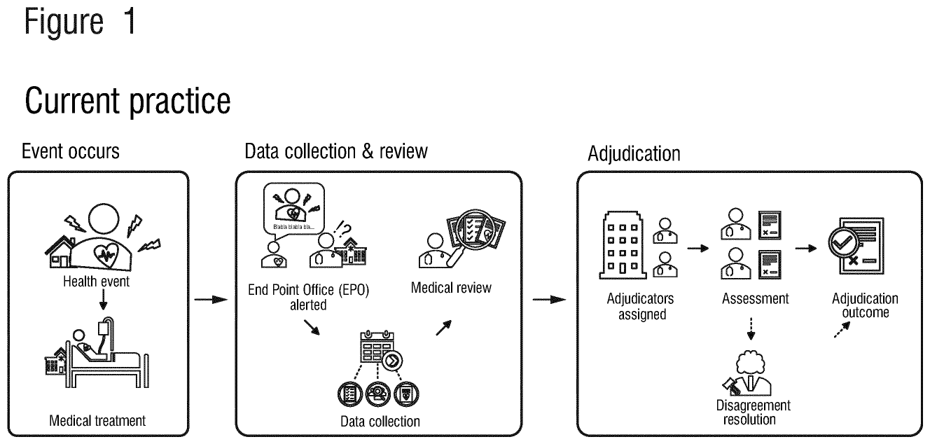

An example of a conventional approach to clinical endpoint adjudication is illustrated in FIG. 1. As can be seen, clinical endpoint adjudication may begin when a health event occurs (e.g. myocardial infarction) that necessitates medical treatment. This causes an alert to be sent to an end point office (EPO) resulting in data collection (e.g. relating to the patient and the circumstances leading up to the event, the medical treatment, any tests performed etc.) and medical review. Once this has been performed adjudication occurs. An expert panel or committee of adjudicators is assigned, and an adjudication assessment is performed. This may either agree with the medical review or disagree, and if necessary, disagreement resolution is performed before an adjudication outcome is decided.

As noted above, this is a time consuming and costly process. There can be a delay in reporting events to an end point office, and further delays in collecting data. The adjudication process can also take a significant amount of time before it occurs and is very time consuming for the committee. Furthermore, for very large-scale multi-centre and multi-country studies it may not always be possible to have the same adjudication panel.

The present inventors have identified a novel solution for addressing these problems. The novel solution seeks to both reduce the number of events sent to medical review and provide automated classification for the bulk of outcome study endpoints. They have managed to perform this by implementing an automated event adjudication process.

Automated Event Adjudication may provide:
Efficient, automated clinical event classification and medical review triage;
Reduce the time to identify clinical events;
A unified, consistent process for classification of clinical events across TAs; and
Proactive identification of events and support for IoT endpoint ID in near real-time.

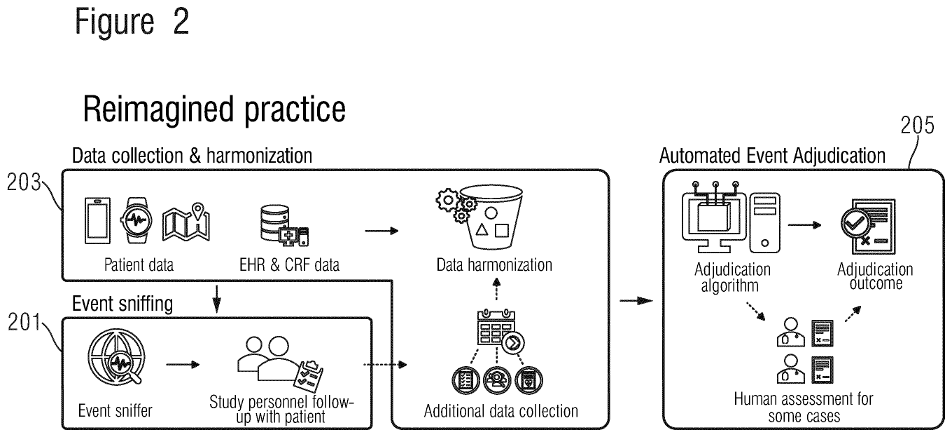

There are three main aspects or modules that have been developed that enable automated event adjudication to occur. These three modules are shown in FIG. 2 and are:
(i) the implementation of an “event sniffer” module 201 that can detect when an event occurs;
(ii) tools for harmonizing and ensuring the quality of data obtained meets the requirements for event adjudication—“Data harmonization and collection” module 203; and
(iii) a machine learning approach to performing the clinical endpoint adjudication itself—“Automated Event Adjudication” module 205.

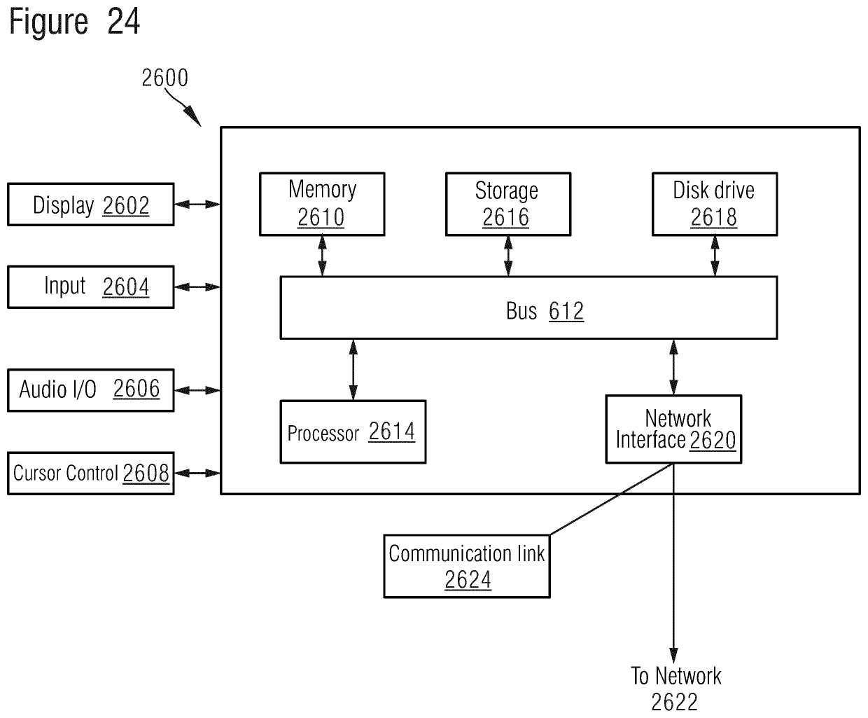

As can be seen from FIG. 2, these three aspects or modules may work together, so that the outputs from the event sniffer 201 and data harmonization and collection module 203 may be used as inputs to the automated event adjudication 205. For example, if the event sniffer module 201 determines that there is a relatively high likelihood (for example, above a selected threshold level of likelihood) that a healthcare event has occurred, the automated event adjudication module 205 may use the results from the event sniffer 201 and the harmonized data output from the data harmonization and collection module 203 to determine an adjudication outcome. It will be understood that these three aspects or modules may be implemented in hardware and/or software either individually or in combination. For example, the modules may be implemented on a computer system 2600 as described below with reference to FIG. 24, for example being stored in memory 2610 or storage 2616 and implemented by processor 2614. However, the modules may also be implemented on respective computer systems. It will also be understood that the modules listed above may be implemented on a remote server, for example operating as a “cloud” and accessible via a telecommunication network such as the Internet. It will be understood that all of the modules may be implemented on the same remote server or may be implemented on different remote servers.

These three aspects will now be discussed in detail.

### Event Sniffer

Historically, investigators have struggled to obtain relevant information about endpoint events (e.g., hospitalizations) in a timely manner in Cardiovascular Outcomes Trials (CVOTs). Additionally, some events are sometimes never reported and thus never known to investigators. Such data gaps and delays negatively impact data quality, the timeliness of endpoint event data collection, and patient experience in trials. To increase the number of detected unplanned hospitalizations as well as speed up the detection of such events, an “event sniffer” has been developed that monitors and analyzes signals in near-real time about patient condition from a variety of data sources.

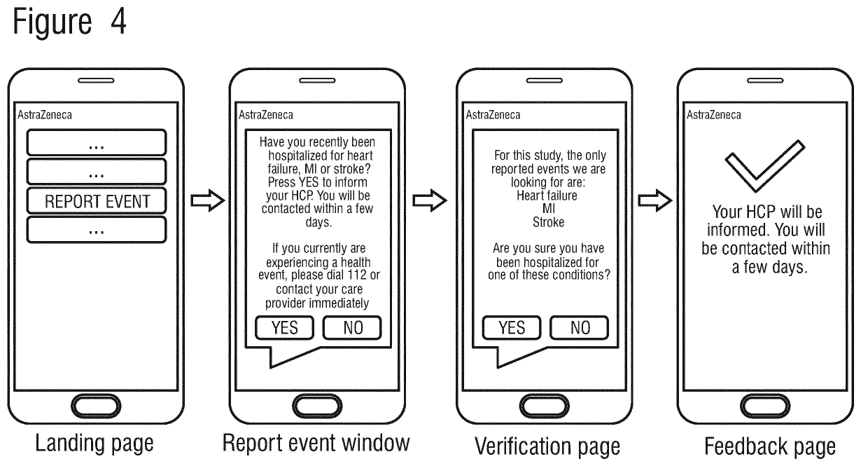

The “event sniffer” is a monitoring system for use in determining whether a healthcare event has occurred for a participant in a clinical trial. The event sniffer can allow a patient to actively report that an event has occurred, for example via the use of an application on their mobile device as shown in FIG. 4. It may combine signals from a variety of sources, and depending on the source of the data signals determine the probability of a healthcare event occurring/having occurred based on parameter(s) of the patient provided by the signal(s) as well as a weighting applied based on the source of the signal(s). For example, signals may be received from patient-connected devices that report a parameter of the patient (such as heart rate, blood pressure, respiratory rate etc.) and a weighting may be applied based on the source of the signal—for example if a known brand or make of heart rate monitor is known to be more reliable then it may be weighted more favorably than a heart rate monitor that is known to be less reliable.

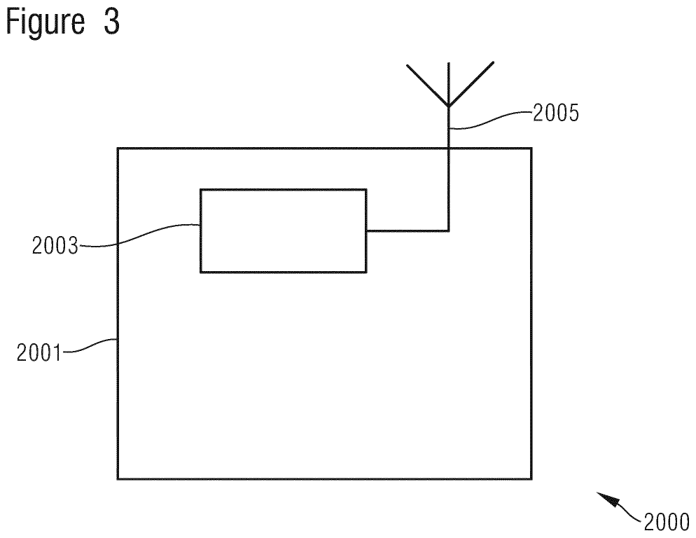

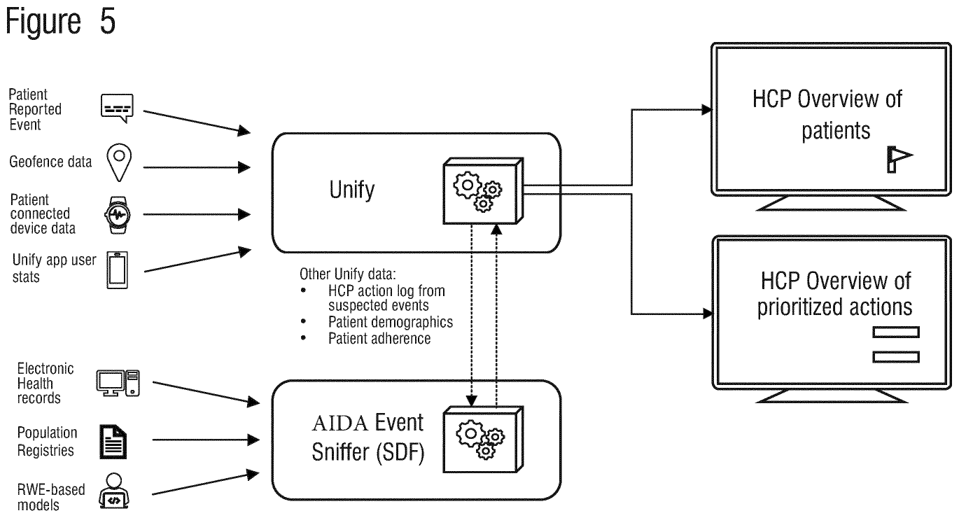

The system may be configured to obtain information indicative of a parameter of the patient from other sources as well—this is shown in more detail in FIG. 3 and explained in more detail below with reference to for example FIGS. 5 to 10, but for example, the system may be configured to obtain location data and determine when a patient visits a medical facility (such as a hospital) by performing a lookup against a database of known medical facility locations and based on the duration of time spent at that location. For example, if the user is at a known location of a medical facility for greater than a selected threshold period of time, then the system may determine that that user is at a medical facility. If it is determined that the user is at a medical facility, the system may be configured to provide a notification or alert to the user (for example, on an app running on the user's mobile device) asking them to confirm whether or not they are at a medical facility and whether or not a healthcare event is or has occurred, as shown in FIG. 4.

Advantages of the event sniffer system are that it is operable to identify clinical events in near real-time independent of clinical sites and medical review.

The event sniffer module 201, receives signals from multiple sources, including from AstraZeneca's® Unify app, Electronic Health Records from health care institutions and/or national registries, and proprietary, internally generated predictive risk algorithms based on patient data available in electronic data capture (EDC). These signals are shown in FIG. 5 and described in more detail below, broken down by source and role that the signal will play within the “event sniffer” capability. FIG. 5 shows a schematic process flow-chart of the functionality of an event sniffer module, with signals from Unify being an example of real world or “real-time” data (for example obtained from the patient in real-time), and signals obtained from the AIDA sniffer being an example of pre-known data that can be obtained from healthcare records and databases.

Signals from Unify:
A. Hospital Geofencing (Trigger Event signal): An algorithm within Unify that leverages smart phone operating system location services to monitor when a patient enters a hospital and stays for a specified amount of time consistent with a CV-related endpoint event (e.g., 18 hours); hospital geofence databases are proprietary datasets; when the geofence rule is triggered, the Unify app asks the patient to confirm they are having a medical event and all metadata is sent to the Unify back end. This source may represent a “Trigger Event” signal, which means the event sniffer system may be triggered to determine whether a healthcare event has occurred when signals from this source are received. It will be understood that a different weighting may be applied to “Trigger Event” signals to “Contextual” signals.
B. Patient-reported event (Trigger Event signal): A Unify app functionality where patients can self-report that they are potentially experiencing an endpoint event; the app also reminds patients once every two weeks to self-report an event if they have not done so; the app sends all self-reported events and associated metadata to the Unify back end. This source may represent a Trigger Event signal.
C. Connected Device Measurements (Trigger Event signal): A Unify app functionality that enables patients to self-administer a measurement using a connected medical device (e.g., blood pressure cuff). The app enables a Bluetooth connection between device and smart phone/tablet. The app then sends a recorded measurement, including metadata, to the Unify back end. Algorithms will assess each measurement for a potential endpoint event associated with measurement data. Algorithms could be built into the app on the “edge” (i.e., occurring in the app on the smart device) or in the Unify back-end, or both. All connected device measurement data and metadata, including signals of potential endpoint events, will be sent by the app to the Unify back-end. This source may represent a Trigger Event signal. Devices that will be available for patients in trials via Unify include, for example:
   a. Omron® Blood Pressure Cuff
   b. Marsden® Weight Scale
   c. MightySat® Rx Pulse Oximeter
D. App User Statistics (Contextual signal): The Unify app will also collect patient user statistics (e.g., time on task, time between log-ins, etc.) which may be used as contextual signals. This means that although trends in user stats may not themselves trigger the event sniffer system to determine that a healthcare event has occurred, they will provide additional, contextual signal about the patient condition at the time a trigger (e.g., from a geofence event).

### Additional Signals:

E. Real World Evidence (RWE)-based predictive models (Contextual signal): Machine learning algorithms that aim to help health care providers (HCP's) prioritize notifications about potential patient events; algorithms are trained using RWD claims data and/or historical AZ data from similar trials. These algorithms may provide contextual signals; they may provide additional, contextual signals about the patient condition at the time of a trigger (e.g., from a geofence event).
F. Electronic Health Records (Trigger Event signal): This data source consists of structured and unstructured electronic health record data received directly or indirectly (i.e., via services lie TriNetX) from medical institutions that are made available to electronic data capture (EDC) about a patient hospital visit. This data provides detailed information about a patient's hospital visit (e.g., discharge report) that provide signals about a potential endpoint event. This signal can consist of raw signal (e.g., data directly from EHR) and/or the result of an AI algorithm that is trained from said EHR data. This source may represent a Trigger Event signal.
G. Population Registries (Contextual signal): This source consists of national registry data made available by some national and/or regional governments about populations, including deaths. This source will provide contextual signals; they may provide additional, contextual signals about the patient condition at the time a trigger (e.g., from a geofence event).

The event sniffer module is configured to orchestrate and combine signals into a single aggregated signal (aka Event Sniffer Dossier), for example following a Trigger Event, and then qualifies and ranks the single aggregated signal as a potential endpoint or healthcare event, all in an automated workflow. See detail below on each of these functions:
Data orchestration and combination: If any Trigger Event occurs (as described above), the system is configured to scan for concurrent (and/or recent) trigger and contextual signals from all other sources and combines them together into a single, aggregated signal, called an Event Sniffer Dossier. The specific data elements for which the event sniffer system scans include: presence of other Event Trigger and/or Contextual signals; the age of all identified Event Trigger and/or Contextual signals.
Event ranking: Additionally, the system is configured to provide an overall signal ranking that is then served back to the Unify system for presentation to a site health care provider (HCP). This ranking, done using a combination of business rules and machine learning that evaluates and weights each available signal in an Event Sniffer Dossier

Two examples of how the event sniffer module 201 may work are now described below by way of example only.
Example 1—low ranking event: A connected device rule triggers (high blood pressure for 1 day), but no other trigger events are present in the patient's dossier (no hospital geofence info; no patient reported events). Additionally, the RWD/RCT predictive risk algorithm indicates that the patient is at low/moderate risk for Myocardial Infarction at the current time. This information is combined in a dossier (an Event Sniffer digital dossier that aggregates all events from different sources and metadata about the patient) and an event ranking score is generated of 0.3, which means that the event is not likely to be a relevant endpoint event.
   Note: score of 0.3 is currently representative and does not reflect an output of a working algorithm
Example 2—high ranking event: The patient self-reports an event via the Unify app. This event is combined with two other trigger signals: a hospital geofencing alert (which had triggered a few hours before) and a connected device business rule (high blood pressure from each of the previous 2 days). Additionally, the RWD/RCT predictive risk algorithm indicates that the patient is at high risk for Myocardial Infarction at the current time. This information is combined in a dossier (the Event Sniffer digital dossier) and an event ranking score is generated of 0.9, which means that the event is highly likely to be a relevant endpoint event.
   Note: score of 0.9 is currently representative and does not reflect an output of a working algorithm
   Note: a previous trigger of connected device and geofence implies that there was an existing score prior to 0.9. The intended functionality is for each new event to “update” the patient's Event Sniffer dossier with relevant information and update the risk score, for ranking.

An example of the event sniffer or monitoring system 2000, that may have the functionality as described above, is shown in FIG. 3. The event sniffer system 2000 may be a monitoring system for determining whether a healthcare event has occurred for a participant in a clinical trial. The system 2001 comprises a communications interface 2005 coupled to a processor 2003. It will be understood that the event sniffer system 2000 may be provided on a portable electronic device (e.g. a smartphone, tablet or laptop) or may be provided remotely, and accessible for example via the cloud. In some examples the system 2001 may have similar or the same functionality and/or components to the system 2600 described below with reference to FIG. 24. In some examples, the system 2001 may further comprise an optional location module, such as GPS module or other means for determining the location of the system 2001.

The communications interface 2005 is configured to receive data signals relating to a plurality of participants from a plurality of sources, wherein the data signals each comprise information indicative of a parameter associated with a participant. The processor 2003 is configured, for each patient, to process each received data signal and apply a first weighting to each data signal based on the source of the data signal. It will be understood that the weighting could be applied to indicate whether the signal is a “trigger event” signal or a “contextual” signal, with a higher weighting being applied to “trigger event” signals than to “contextual” signals.

The processor 2003 is further configured to make a determination of the probability of a healthcare event occurring based on at least one of: (i) the data signal indicating that a parameter associated with a patient exceeds a selected threshold for that participant, and (ii) the first weighting exceeding a selected trigger threshold.

For example, the selected threshold may for example be a distance to a medical facility (such as a hospital) being less than 100 m, or heart rate exceeds 160 bpm, or respiratory rate exceeds 30. The selected threshold may also be based on other parameters of the patient—for example, the selected threshold may vary as a function of age, such that the heart rate threshold for example is lower for someone who is older than for someone who is younger.

In some examples the processor 2003 may be configured to make a determination of the probability of a healthcare event occurring based on both (i) the data signal indicating that a parameter associated with a patient exceeds a selected threshold for that participant, and (ii) the first weighting exceeding a selected trigger threshold. In this way, for example, the processor 2003 may only make a determination if a “trigger event” signal is received, for example if the patient is within a selected distance of a medical facility. In this way, the processor 2003 may be configured to make a determination of the probability of a healthcare event occurring based on a plurality of data signals with at least one data signal indicating at least one of (i) that a parameter associated with a patient exceeds a selected threshold and that (ii) a weighting of that data signal exceeds a selected threshold. For example, the processor 2003 may be configured to combine the signals into the Event Sniffer Dossier as described above.

The processor 2003 may be configured to make a determination that a healthcare event has occurred based on the determined probability exceeding a selected threshold (for example, a probability greater than 50%, greater than 70%, greater than 90%), and in the event that the processor 2003 determines that a healthcare event has occurred, the processor 2003 is configured to provide a notification to a user of the monitoring system (for example, as shown in FIG. 5), and wherein the monitoring system 2000 is configured to rank the notifications based on the determined probability of the healthcare event occurring, for example based on a patient's response to the notification.

The processor 2003 may be configured to make a determination of the type of healthcare event based on the source of the data signal and the indication of the parameter associated with the participant. In some examples the processor 2003 is configured to rank the notifications based on the determined type of healthcare event—for example events that are determined to be more severe may be ranked higher. In some examples the processor 2003 is configured to rank the notifications based on the known health of the participants.

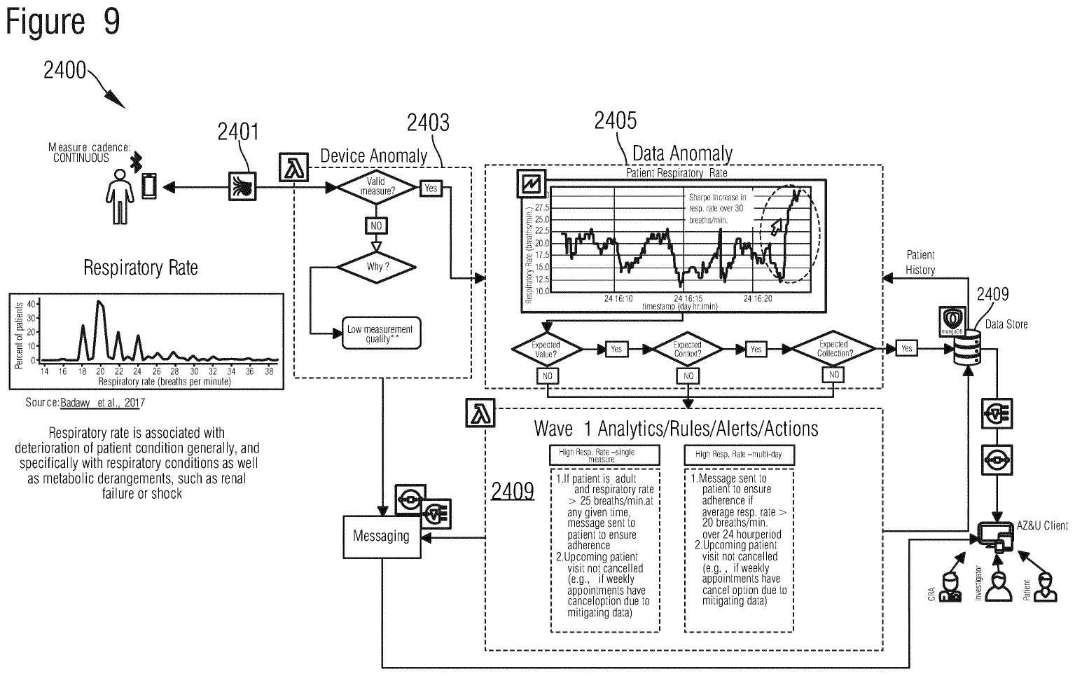

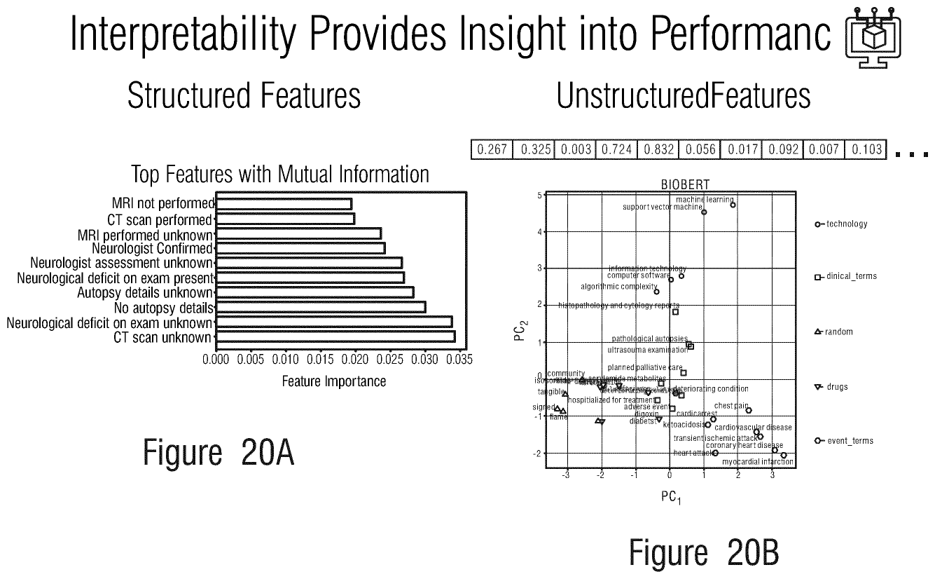

In some examples when the processor 2003 makes a determination of the probability of a healthcare event occurring based on a received data signal indicating that a parameter has exceeded a selected threshold, the processor 2003 reviews information indicative of previous values of that parameter associated with the participant in a selected time interval preceding the determination. The selected time interval may, for example, vary depending on the relevant parameter. For example, the processor 2003 may review blood pressure over the course of the last week, but heart rate over the course of the previous 12 hours. The processor 2003 may only review previous values of that parameter if that parameter has exceeded a selected threshold, but in other examples may review previous values of a parameter or parameters if a different parameter has exceeded a selected threshold. Doing this may help to determine not only the type of healthcare event but may also provide a degree of verifiability. It may help to distinguish between measurement error (e.g. missing data/poor data quality) and a potential safety event (e.g. single point anomaly of elevated respiratory rate, or trend anomaly of weight gain), as described in more detail below with reference to FIGS. 9 and 20.

If it is determined that the probability of an event having occurred exceeds a selected threshold, then the processor 2003 may be configured to make a determination as to whether more information is needed from the patient, and in the event that more information is needed, then a notification is provided to the health care provider/system administrator to contact the patient, for example via the app as shown in FIG. 4. For example, the processor 2003 may require information indicative of a minimum set of distinct parameters from a patient, and the processor 2003 may be configured to determine if any of that information is missing and/or has not been obtained recently enough, for example within a selected window of time of the determination.

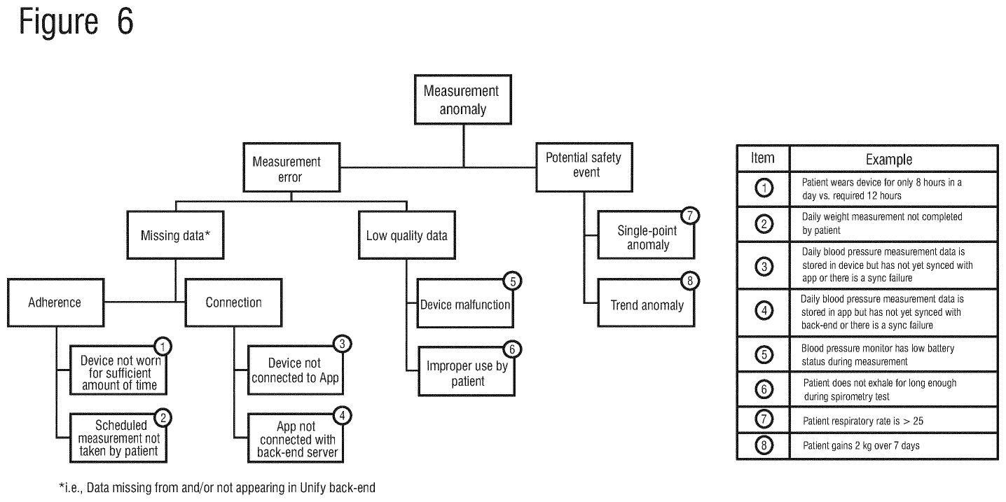

The processor 2003 may also be configured to make a determination of the reliability of the data signal, and to apply a second weighting based on the reliability of the data signal. The processor 2003 may be configured to make a determination of the probability of a healthcare event occurring based on the data signal indicating that a parameter associated with a patient exceeds a selected threshold and the first and second weightings. The reliability of the data signal may be determined from metadata obtained with the signal, for example indicating that the device from which the signal was obtained had a low battery or poor connection, or if data has not been uploaded recently. The determination of the reliability of the signal will be discussed in more detail below with reference to FIGS. 6 to 10.

The processor 2003 may additionally or alternatively be configured to make a determination of the probability of a healthcare event occurring for a participant based on any previous determined probabilities of an event occurring for that participant.

It will also be understood that the monitoring system 2000 shown in FIG. 3 may be operable to determine whether a healthcare event has occurred based solely on location data and the duration of the patient's stay at a particular location. For example, the communications interface 2005 may be configured to receive data signals relating to a plurality of participants from a plurality of sources, wherein the data signals each comprise information indicative of a location associated with a participant. For each participant, the processor 2003 may be configured to process each received data signal and make a determination of the probability of a healthcare event occurring for a participant based on (i) the proximity of the participant to a known healthcare centre and (ii) the duration of the participant's proximity to the known healthcare centre. In the event that the processor 2003 determines that the probability of a healthcare event occurring exceeds a selected threshold, the processor 2003 is configured to send a notification to the participant requesting confirmation from the participant that a healthcare event has occurred (for example, as shown in FIG. 4).

As noted above, the processor 2003 may be configured to determine the reliability of the data signal. For example, the processor 2003 may attempt to find evidence of minimal variance in measurements from the same device by same subject at the same time for an endpoint of interest. An example way of performing this may be:
Take multiple, consecutive measurements of blood pressure using connected device+app (Bluetooth), with data sent to back-end storage
Conduct at least 15 measurements within 30-minute window; repeat experiment as needed

Success if:
Intraclass correlation coefficient of measures >0.9

Ways of dealing with potential anomalies are illustrated in FIGS. 6 to 10, and in particular, for determining whether the event is an anomaly or may be indicative of a healthcare event having occurred. The anomaly may be an anomaly in the device which obtained the data (a device anomaly) or may be an anomaly in the data itself (a data anomaly). FIG. 6 shows the steps the processor 2003 may perform to determine whether a potential anomaly has occurred. When the processor 2003 first receives data via the data signals, it determines whether there is a potential measurement error and/or a potential safety event. In determining whether there is a potential measurement error, it determines whether there is missing data and/or low-quality data. If there is missing data, it determines whether the data is missing by virtue of adherence (e.g. the patient not wearing a measurement device for a sufficient period of time or a scheduled measurement not being taken by a patient) or by virtue of a poor connection (e.g. the measurement device was not connected to the measurement app and/or the app is not connected with the back-end server). If there is low quality data, the processor 2003 seeks to determine if the device has malfunctioned and/or if it was improperly used by the patient. A potential safety event may be determined, for example, when there is a single point anomaly and/or a trend anomaly, for example that does not accord with other data indicative of other parameters of the patient. The potential safety event, however, may be indicative of a healthcare event and so in some examples a potential safety event may trigger a determination being made as to whether a healthcare event has occurred, for example that makes use of other data indicative of other parameters of the patient, such as from the Event Sniffer Dossier.

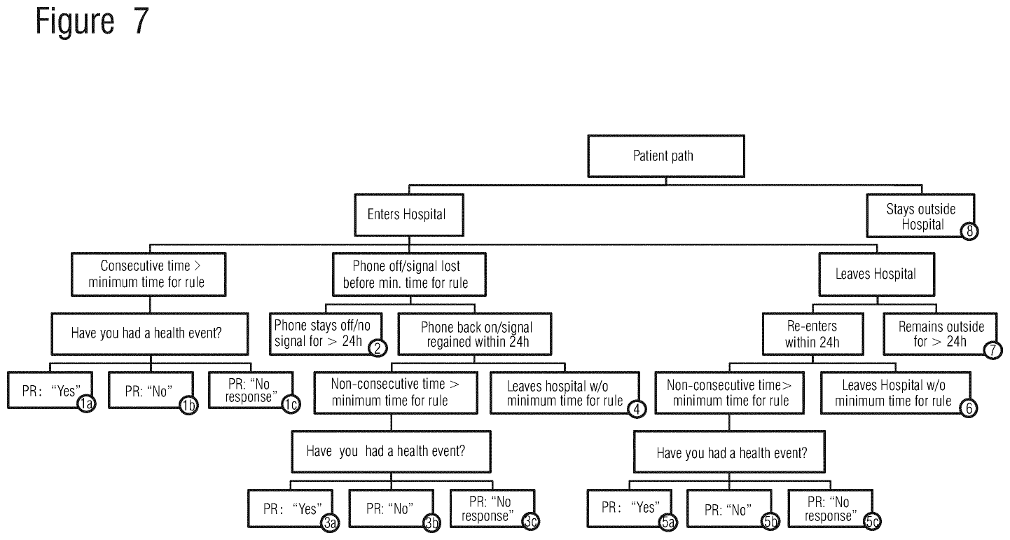

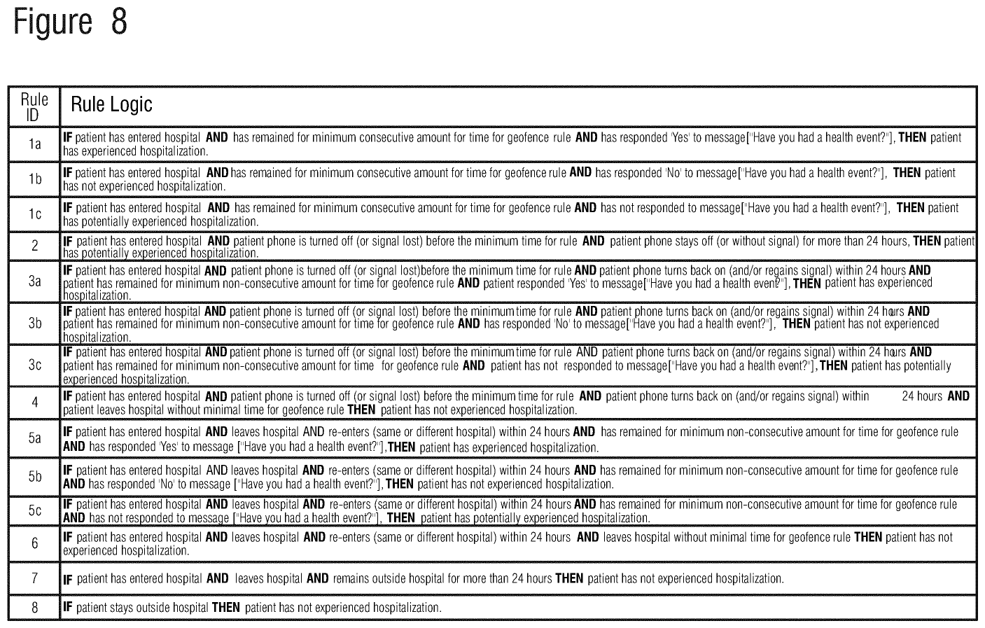

FIGS. 7 to 10 illustrate ways in which the system 200 may also seek to address anomalies and inconsistencies in the data in examples where the monitoring system 2000 is operable to determine whether a healthcare event has occurred based on location data and the duration of the patient's stay at a particular location. FIG. 7 shows a process flow chart as to how a determination is made, and FIG. 8 shows the rules that are used to determine whether a healthcare event has occurred or not. For example, as can be seen in FIGS. 7 and 8, if the patient has been in a hospital for a consecutive period of time greater than a selected minimum threshold period of time, an indication may be provided to the patient (as shown in FIG. 4) on their app to ask the patient to confirm whether they are having or have had an event. FIGS. 7 and 8 also show what happens if the patient's phone is off/loses signal and/or if the patient leaves the hospital. In both cases, if the patient returns and/or if the signal is regained within 24 h and the patient is in the hospital for a non-consecutive period of time greater than a threshold period of time, the patient is once again asked to confirm whether they are having or have had an event. If the patient confirms, then the system determines that an event has occurred and allocates a high probability (e.g. above 90%) to the event. If the patient replies in the negative indicating that they have not had an event, then the system determines that an event has not occurred and allocated a low probability (e.g. less than 10%) to the event. If the patient does not reply, then the system determines that the patient may have potentially had an event and allocated an appropriate probability (e.g. 50%). Of course, these probabilities may be adjusted by the system for example based on other parameters obtained from the patient and their respective data signals and weightings that may be obtained from the Event Sniffer Dossier.

Examples of parameters that may be obtained from a patient via the data signals include:
Age
BMI
Height
Systolic Blood Pressure
Creatinine Clearance (mL/min)
Glomerular Filtration Rate C-G (mL/min/1.7)
Glomerular Filtration Rate MDRD (mL/min/1.7)
Alkaline Phosphatase (IU/L)
Apolipoprotein A1 (g/L)
Apolipoprotein B (g/L)
Glucose (mmol/L)
Hemoglobin (g/L)
Lymphocytes (10\hat{ }9/L)
Lymphocytes/Leukocytes (%)
Neutrophils (10\hat{ }9/L)
Platelets (10\hat{ }9/L)
Urate (umol/L)
Leukocytes (10\hat{ }9/L)
Whether the patient is taking Glyceryl Trinitrate and/or in what dose
Whether the patient is taking Furosemide and/or in what dose
Whether the patient is taking Heparin and/or in what dose

FIG. 9 shows an example process flow chart of a method 2400 of determining whether an anomaly or a healthcare event has occurred. At step 2401 data (in this example, continuous respiratory rate) is received from a patient. In the example shown the data may be received from a wearable device worn by the patient and transmitted wirelessly (e.g. via Bluetooth®) to a handheld device, which may then forward the data onto a remote monitoring system operating in the cloud. At step 2403 the received data is processed to determine whether there is a device anomaly. This may comprise, for example, reviewing any metadata provided with the data indicating, for example, that the device from which the data was received had a low battery, a poor connection, or did not obtain an accurate reading. If the data is determined to contain a device anomaly, the process ends there and the data is not used. By contrast, if the data is determined not to contain a device anomaly, the data is then analysed at step 2405 to determine if there is a data anomaly, indicative of either a healthcare event or of an anomaly in obtaining the data. This analysis is performed by comparing a recent value or set of values (e.g. obtained over a selected time interval so as to determine a general trend) to previous and/or expected values of data for that patient. For example, the method may comprise determining expected values for a resting respiratory rate for a patient given their age and other underlying health conditions, and a comparison may be made between the received values and the expected values. The method may do this by obtaining 2409 patient history from a data store. If the comparison indicates a divergence greater than a selected threshold level of divergence, this may indicate a potential healthcare event and/or data anomaly. In the present case, the data indicates a sharp increase in respiratory rate over 30 breaths/minute over a recent time period. This may indicate a potential healthcare event.

In the event that a potential healthcare event and/or data anomaly is determined, a set of rules/actions are then applied at step 2407. These rules may comprise providing an indication or notification to the user to ask them if there is a healthcare event and to ensure that they are correctly obtaining their data (e.g. correctly using the respiratory rate monitor) in the correct manner, as illustrated for example in FIG. 4. If a user responds by indicating that they are using the device (in this case the respiratory rate monitor) in the correct manner, then the system may determine that there is a high likelihood that a healthcare event has occurred. In the present case, because the data indicates a sharp increase in respiratory rate over 30 breaths/minute over a recent time period, a notification is sent to the user to ask them to confirm that they are correctly using the device (respiratory rate monitor). This notification may include instructions as to how to correctly use the device, which in this example is the respiratory rate monitor.

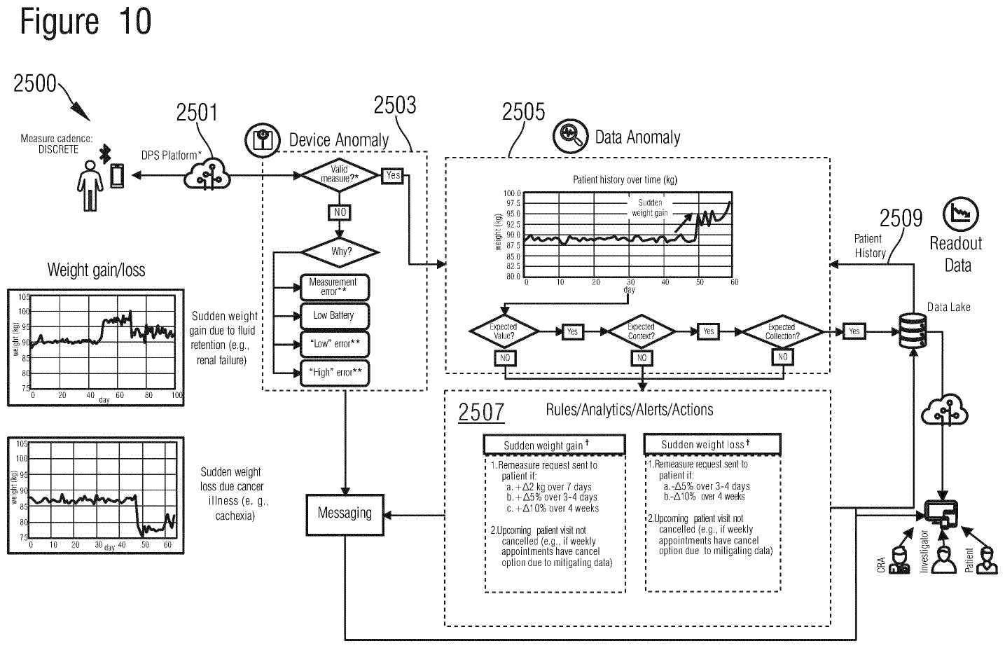

FIG. 10 shows another example process flow chart of a method 2500 of determining whether an anomaly or a healthcare event has occurred. At step 2501 data (in this example, discrete weight data) is received from a patient at discrete time intervals. In the example shown the data may be received by the patient entering their weight into an app operating on a handheld device may then forward the data onto a remote monitoring system operating in the cloud, or via a “smart” set of scales which automatically uploads the data to the remote monitoring system. At step 2503 the received data is processed to determine whether there is a device anomaly. This may comprise, for example, reviewing any metadata provided with the data indicating, for example, that the device from which the data was received had a low battery, a poor connection, or did not obtain an accurate reading. If the data is determined to contain a device anomaly, the process ends there, and the data is not used. By contrast, if the data is determined not to contain a device anomaly, the data is then analysed at step 2505 to determine if there is a data anomaly, indicative of either a healthcare event or of an anomaly in obtaining the data. This analysis is performed by comparing a recent value or set of values (e.g. obtained over a selected time interval so as to determine a general trend) to previous and/or expected values of data for that patient. For example, the method may comprise determining expected values for a resting respiratory rate for a patient given their age and other underlying health conditions, and a comparison may be made between the received values and the expected values. The method may do this by obtaining 2509 patient history from a data store. If the comparison indicates a divergence greater than a selected threshold level of divergence, this may indicate a potential healthcare event and/or data anomaly. In the present case, the data indicates a sharp increase in weight over a recent time period (e.g. over a series of consecutive days). This may indicate a potential healthcare event.

In the event that a potential healthcare event and/or data anomaly is determined, a set of rules/actions are then applied at step 2507. These rules may comprise providing an indication or notification to the user to ask them if there is a healthcare event and to ensure that they are correctly obtaining their data (e.g. correctly using the scales) in the correct manner. If a user responds by indicating that they are using the device in the correct manner, then the system may determine that there is a high likelihood that a healthcare event has occurred. In the present case, because the data indicates a sharp increase in weight over a recent time period, a notification is sent to the user to ask them to confirm that they are correctly using the device and/or to request that the measurement is repeated. If the measurement is repeated and the same or a similar (e.g. to within a selected threshold) result is obtained, then the system may determine that a healthcare event has occurred.

### Data Harmonization

As noted above, to perform effective clinical endpoint adjudication, a reliable and robust data set or adjudication dossier is required. A problem with this is that due to the nature of such clinical studies and the fact that they may be performed over a large geographical area, the way in which data is obtained and recorded can vary dramatically. The present inventors have therefore developed a data harmonization and collection system 203 that re-engineers the clinical event dataflow to support diverse data types and extraction of meaningful events across data types and streaming data.

In more detail, the data harmonization and collection system 203 aims to apply machine learning to prepare and provide Machine Learning (ML) ready data sets.

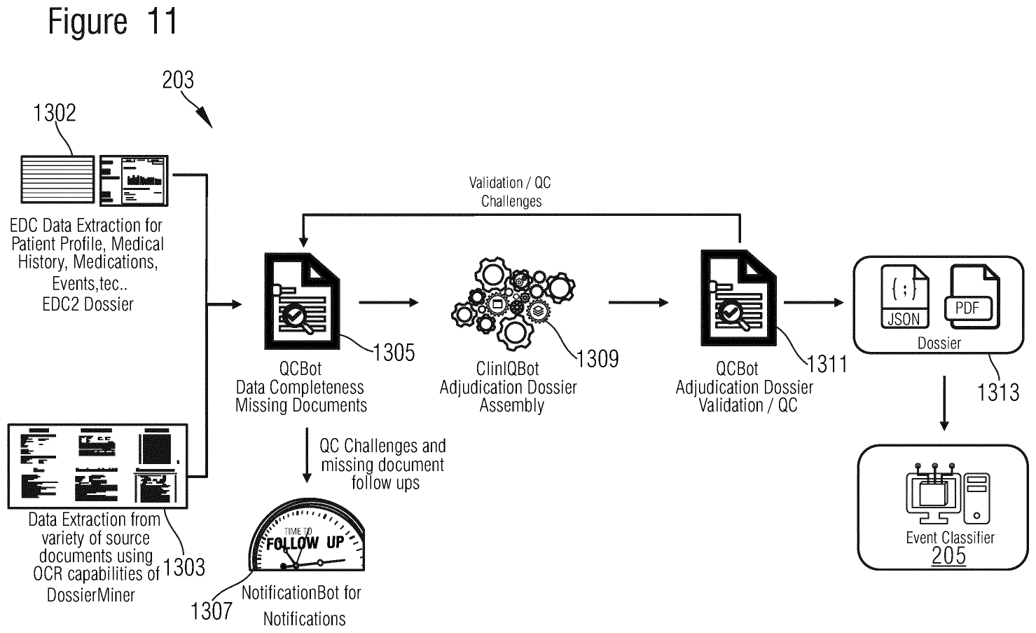

In order to achieve the above objectives, the data harmonization and collection system 203 may comprise a number of modules illustrated schematically in FIG. 11:
ClinIQBot 1309: A software bot driven intelligent automation for assembly, curation and quality control of clinical dossiers.
EDC2Dossier 1302 and DossierMiner 1303: Configurable clinical data extraction tools from source documents. EDC2Dossier 1302 is for extracting structured data from underlying Electronic Data Capture (EDC) system, and DossierMiner 1303 is for data extraction from source documents.
NotificationBot 1307: A suite of microservices to provide targeted capabilities for study configuration, notifications, digital dossier generation for ongoing studies, dossier assembly and for quality control.

It will be understood that the data harmonization and collection system 203, and the modules listed above, may be implemented in hardware and/or software either individually or collectively. For example, the modules may be implemented on a computer system 2600 as described below with reference to FIG. 24, for example being stored in memory 2610 or storage 2616 and implemented by processor 2614. However, the modules may also be implemented on respective computer systems. It will also be understood that the modules listed above may be implemented on a remote server, for example operating as a “cloud” and accessible via a telecommunication network such as the Internet. It will be understood that all of the modules may be implemented on the same remote server or may be implemented on different remote servers.

Clinical Event Adjudication (CEA) is a critical component of clinical trials and uses Adjudication Dossier (AD) as the key input for making its adjudication decisions. Current processes to assemble an AD is complex, time intensive and involves many sub processes. Some of these sub processes include data extraction, document collection, quality control, site follow up, etc. The data collection process itself for assembling an AD could take over 30 days at times. The combination of manual processes and the time they take, presents an ideal opportunity to innovate and automate the dossier creation workflow, which could lead to many benefits in terms of time, quality and process simplification.

An AD is composed of data from both structured and unstructured sources, as shown in Ft. The structured data includes information such as patient profile, medical history, medications that comes from the underlying EDC system. The standards and requirements for capturing this data may differ for each study as each study is unique. An EDC2Dossiers module 1302 can create digital dossiers based on the EDC data. The unstructured data may come from documents such as discharge summary, death certificate, autopsy report, etc. The format of these documents differs for each country and sometimes for each hospital that is participating in the trial. The DossierMiner module 1303 also creates digital dossiers based on the source documents.

The outputs of the DossierMiner module 1303 can be validated with a set of quality rules and managed by the QC Service. A digital dossier service will combine these outputs to create the complete Virtual Adjudication Dossier 1313. The notifications or communications that are needed to resolve any missing data or documents issues will be managed by the NotificationBot module 1307.

The ClinIQBot module 1309 is an end-to-end automation workflow for assembling Virtual Adjudication Dossiers 1313 by integrating the EDC2Dossiers module 1302, DossierMiner module 1303, and the NotificationBot module 1307. FIG. 11 represents a high-level view of the end-to-end automation workflow ClinIQBot 1309, which, when deployed has the potential to accelerate adjudication processes in clinical trials and set new industry standards for applying intelligent automation to augment clinical trials.

The EDC2Dossiers module 1302 focuses on automating the process to extracting and processing the structured data from completed clinical trials from EDC system. It provides configurable, re-usable pipeline to assemble all the relevant structured EDC data into a digital dossier to drive event adjudication or event detection for a given event in a clinical trial. Digital dossiers are essentially the machine learning ready clinical trial data in json format with a purpose to enable better performing event classification or event detection algorithms.

In summary, the DossierMiner module 1303 focuses on extracting the data from unstructured source documents, for example in PDF format. It is a re-usable tool to extract data from free text, tables, and images that exist in these documents and can convert them into ML ready data in json format using the state-of-the-art ML & Optical Character Recognition (OCR) technologies, such as Tesseract, and as described in more detail in a paper by Noam Mor and Lior Wolf of The School of Computer Science Tel Aviv University, “Confidence Prediction for Lexicon-Free OCR” 28 May 2018, which is hereby incorporated by reference in its entirety. For example, the calculation of the Tesseract confidence metric is based on comparing it with a character prototype and calculating a distance metric from this representation.

In addition to extracting data, DossierMiner 1303 also assesses the quality extraction quality for each word, table, image and page and can add these quality confidence levels to the json files. These json files will be combined with the digital dossiers created by the EDC2Dossiers module 1302 to assemble the complete Virtual Adjudication Dossier 1313, which will be the key input for the automated event adjudication system 205 (the “event classifier”).

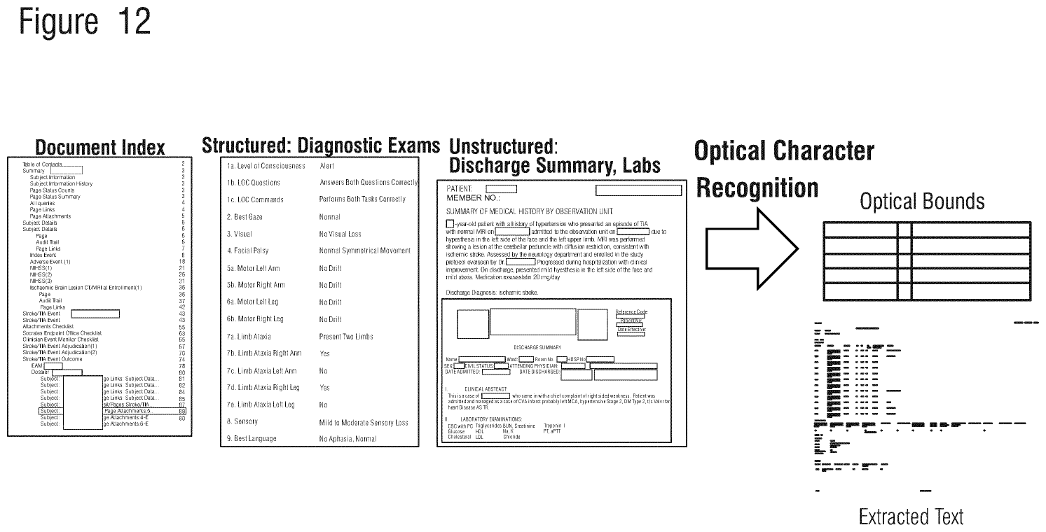

In more detail, the DossierMiner module 1303 is therefore operable to execute a computer-implemented method of harmonising and collating data from a plurality of healthcare-related sources for clinical trial endpoint adjudication. The method comprises analysing each data source to determine whether data held by the data source comprises structured and/or unstructured data. In the event that the data comprises unstructured data, as shown in FIG. 12 optical character recognition (for example, Tesseract) is performed on a region or regions of the data not already in a machine-readable format. A confidence score is attributed as an attribute to the data based on at least one of (i) the data source, and (ii) a determined confidence based on the optical character recognition process. This may be done by performing the method of Noam Mor and Lior Wolf as described above. The confidence score may be attributed on a global level, or the data source may be broken down into regions and a confidence score attributed to each region.

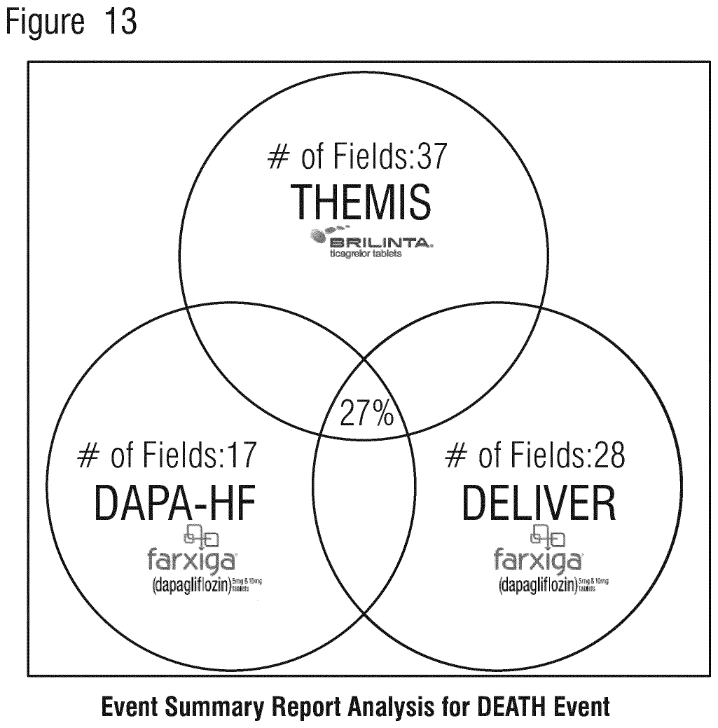

A feature analysis is then performed on the data to extract features from the data, and the extracted features are mapped to a predefined set of features. Mapping is important because it ensures the extracted data matches exactly the same information that is used by the Adjudication Committee. For example, it may be based on what information is available in the Adjudication Dossiers of the historical studies. This is illustrated in FIG. 13, where it can be seen that different clinical studies/trials may have a different number of fields to which data can be recorded, and these fields may not be common to all trials. For example, as shown in FIG. 13, the DAPA-HF trial has 17 fields, the THEMIS trial has 27 fields and the DELIVER trial has 28 fields. There is only a 27% overlap of these fields between the three trials. It is therefore important that the features from each trial are mapped to a common set of features so that the adjudication can be performed on a common dataset.

Once this is done, the mapped extracted features may be published in a json format for use by a machine learning model in performing the clinical trial endpoint adjudication (as will be described in more detail below), wherein the confidence score is an attribute of the feature. In some examples the publication may be performed only when the confidence score is above a selected threshold, for example so that only mapped extracted features whereby there is a relatively high degree of confidence are used.

Performing a feature analysis on the data to extract features from the data may further comprise checking for and removing any duplicated, inconsistent or inapplicable features. For example, inapplicable features may be features that are irrelevant to the event being adjudicated by the model.

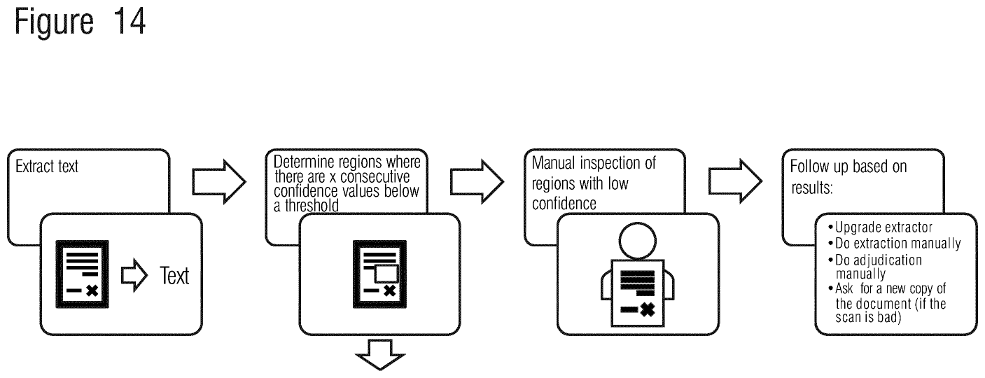

In some examples, if the confidence score is low (for example below a selected threshold), the DossierMiner module 1303, may ask the user to manually review that source of data, as shown in FIG. 14. This may occur, for example, by the DossierMiner module 1303 determining that there a number of regions with consecutively low confidence scores, and the DossierMiner module 1303 may ask the user to manually review those regions. This may be done for example by sending a notification to a user, for example with an indication or image of the regions that need to be manually reviewed.

It will be understood that the features that are need for clinical trial endpoint adjudication may vary, and that they may be based on different endpoints relevant to that clinical trial (e.g. hospitalization, myocardial infarction, death etc.). Accordingly, in some examples the DossierMiner module 1301 may be configured to obtain a set of features needed for a clinical trial endpoint adjudication, wherein the set of features needed is based on the endpoint, and compare the features obtained from the plurality of data sources with the set of features needed for a clinical trial endpoint adjudication to determine whether any features are missing or incomplete. In the event that it is determined that any features are missing or incomplete, the DossierMiner module 1303 may be configured to provide a notification to a user that features are missing, the notification providing an indication of the missing or incomplete features.

In some examples the DossierMiner module 1303 may perform named-entity recognition on the data (as will be described in more detail below with reference to the “Automated Event Adjudication” module 205) prior to performing a feature analysis on the data and selecting formal event characteristics that are relevant to the predefined set of features.

The DossierMiner module 1303 may further be configured to obtain a set of features that should be provided by a data source, and determine whether any features are missing for that data source, and in the event that features are missing for that data source, provide a notification to a user that features are missing. This may advantageously allow complete and accurate adjudication dossiers to be prepared to improve the performance of the adjudication process.

### Classification & Adjudication

The clinical endpoint event adjudication process exists due to variability in clinical site investigator decisions around certain classes of clinical events, for example Major Adverse Cardiovascular Events (MACE). In global clinical trials, site investigators are not always trained in the relevant clinical field (e.g. cardiology) and differences in interpretation of events such as Cardiovascular Death (CVD) and Heart Failure Hospitalization (HF) can introduce quality issues around event reporting. The conventional solution to this is clinical event adjudication, where a committee of trained clinicians assigns multiple clinicians to evaluate a clinical event that occurs in a trial until a consensus is reached as to the type of event. These events are well defined in a charter established during the design of a study and clear criteria around what constitutes an event are outlined. Clinical adjudicators review adjudication dossiers, a collection of structured data such as clinical measurements and unstructured data in the form of medical source documents such as hospital discharge reports, to make a determination of event type.

As a solution to this time consuming and resource-intensive process, the present inventors have developed the automated event adjudication module 205. The automated event adjudication module 205 is an end-to-end process using machine learning algorithms that may be trained on prior clinical adjudicator decisions as ground truth to evaluate adjudication dossiers and determine clinical endpoint events such as CVD and HF. The approach can effectively provide a Quality Assurance (QA) check on Site Investigator decisions around specific events.

The automated event adjudication module 205 takes data from clinical trials that is specified based on the clinical event being adjudicated and applies a machine learning algorithm or suite of algorithms to output a report and recommendation on the classification of a clinical event.

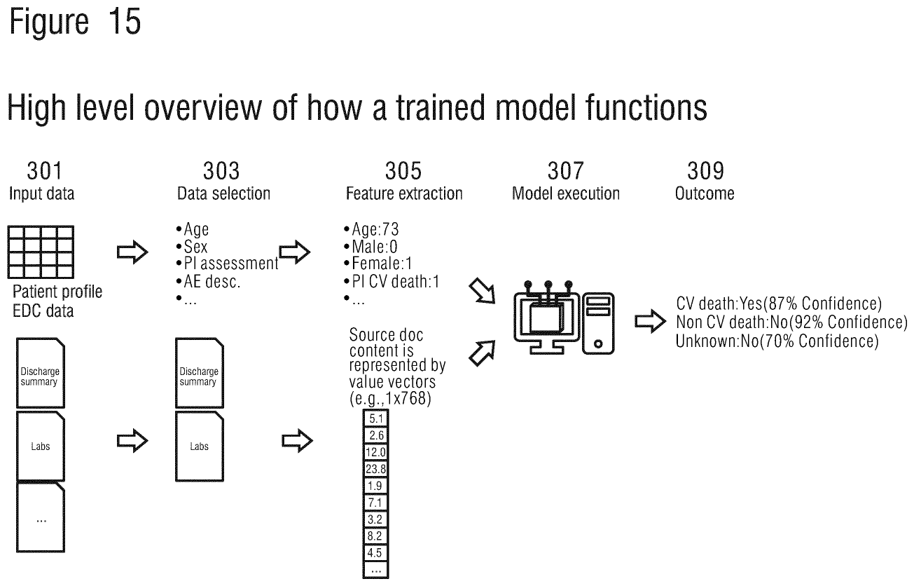

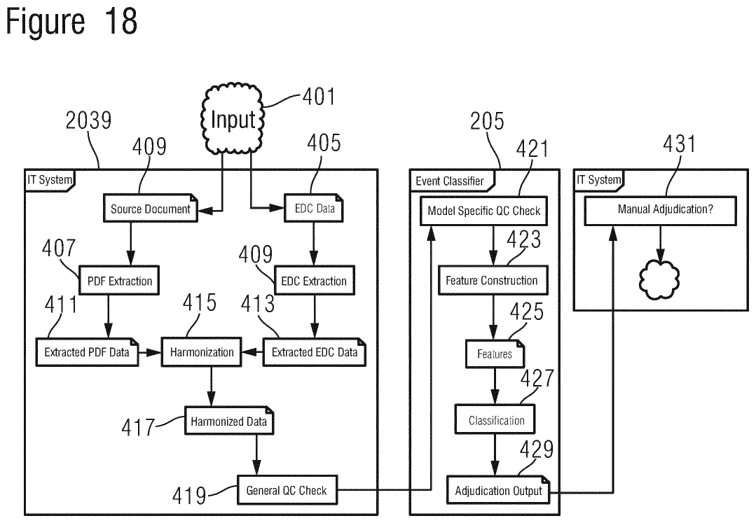

FIG. 15 provides a high-level overview of the steps involved when deploying the automated event adjudication module 205. At step 301, the system receives input data. The data the module receives or uses can be broadly categorized as structured data (sourced from clinical databases) and unstructured data (clinical freetext with no structure or schema encompassing a broad array of clinical documents specified on a per-endpoint event basis). The data inputs can be obtained from the data harmonization and collection module 203 as described above, and as will be described in more detail below with reference to FIG. 18.

At step 303, relevant data is selected, and at step 305 features are extracted from that data. To do this, in the example shown clinical freetext is transformed using a number of known machine learning methods including (but not limited to) bert, biobert, fasttext and named entity recognition (NER) with performance on a given feature set evaluated downstream of the classification task.

At step 307 the model is executed to obtain at step 309 an outcome of a likelihood of an event having occurred.

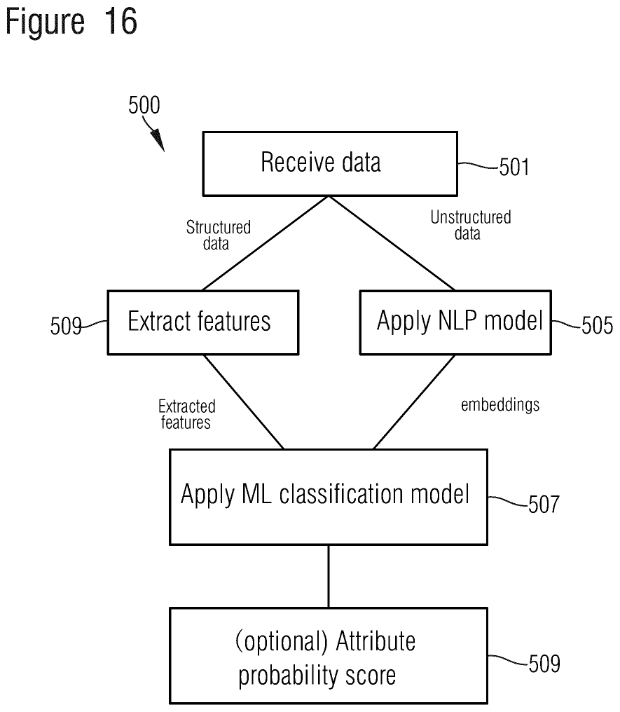

FIG. 16 shows a flow chart of an example computer-implemented method 500 for performing clinical endpoint adjudication. It will be understood that the computer-implemented method may be implemented on a computer system 2600 as described below with reference to FIG. 26, for example being stored in memory 2610 or storage 2616 and implemented by processor 2614.

At step 501 the method comprises receiving data from a plurality of healthcare-related data sources. The data sources may comprise structured data sources and unstructured data sources. Optionally the method comprises the step of analysing each data source to determine whether data held by the data source comprises structured and/or unstructured data. However, it will be understood that the data itself may have an identifier/metadata indicating its source and so this analysing step may not be required.

At step 503, in the event that the data comprises structured data, the method comprises extracting features from that data. At step 505, in the event that the data comprises unstructured data, the method comprises applying a natural language processing model to the unstructured data to obtain embeddings relating to features in the unstructured data. The natural language processing model may include, for example bert, biobert and/or fasttext. In some examples a combination of natural language processing models may be used. The natural language processing models may be pre-trained, for example on clinical datasets. For example, applying a natural language processing model comprises applying a plurality of natural language processing models, comprising a first specialised model trained on text available from the data sources, with a second general model trained on Wikipedia. In some examples, applying a natural language processing model comprises applying BIObert (a model pretrained on biomedical text) which converts into a 768 dimension vector from input biomedical free text and a modified version of fasttext, which combines a specialised model trained on the available free-text with a generalised model trained on Wikipedia which converts both to respective 300 dimension vectors.

In the event that the data comprises unstructured data, in some examples the method further comprises applying a named-entity recognition model to the unstructured data to obtain formal event characteristics from the unstructured data, and applying the machine learning classification model to the formal event characteristics obtained via the named-entity recognition model.

At step 507 the method comprises applying a machine learning classification model to the embeddings from the unstructured data and the features extracted from the structured data to classify whether a healthcare event has occurred based on the embeddings and the features extracted from the structured data. The machine learning classification model may include, for example, Random Forest, Linear SVM or XGBoost.

Optionally, the method comprises step 509 of attributing a probability score as an attribute to the classification, wherein the probability score provides an indication of the likelihood of the event having occurred. For example, the method may further comprise providing a notification to a user to review classifications where the probability score is less than a selected threshold. In this way, only a subset or selection of the events may need to be reviewed by an adjudication committee, thus greatly improving the speed at which adjudication can be completed.

For example, a confidence score may be attributed as an attribute to the data based on at least one of (i) the data source, and (ii) a determined confidence based on an optical character recognition process applied to the unstructured data, and the confidence score may be used as a weighting by the machine learning classification model. The method may comprise excluding data having a confidence score below a selected threshold.

Extracting features from the data and applying a natural language processing model to the unstructured data to obtain embeddings relating to features in the unstructured data may comprise obtaining a predefined set of features for use in the clinical endpoint adjudication and mapping the extracted features and/or embeddings to the predefined set of features, and discarding features and/or embeddings that do not relate to the predefined set of features.

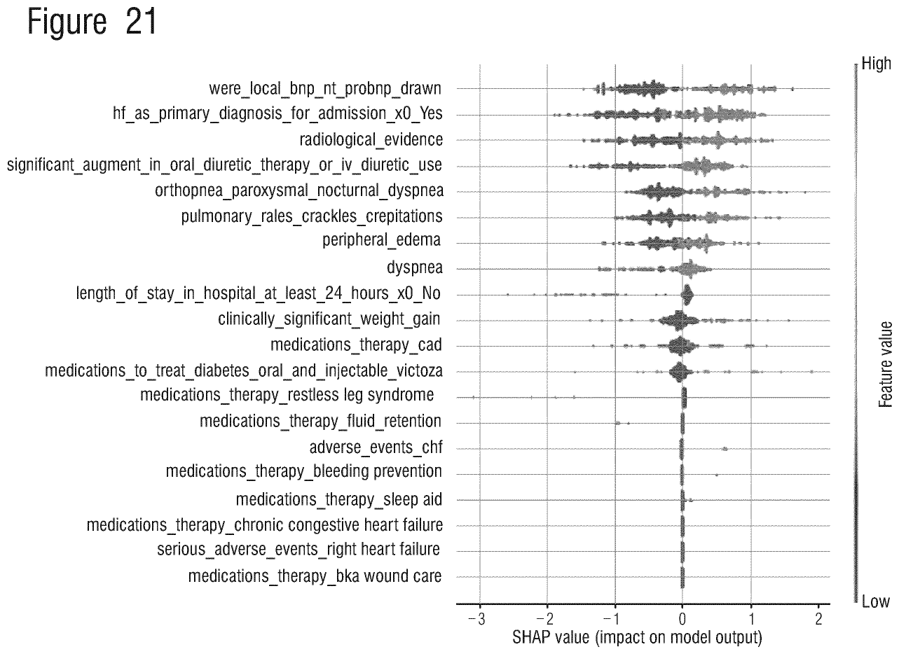

As will be described in more detail below with reference to FIGS. 20A, 20B and 21, the machine learning classification model may provide a ranking of the importance of the features involved in classifying whether a healthcare event has occurred. For example, providing a ranking of the importance of features may comprise determining the SHAP value for each feature and/or applying a local surrogate model to the machine learning classification model to determine the relative contribution of each feature to the classification.

In some examples the method 500 is only performed when the amount of data available exceeds a selected threshold. In some examples the method 500 is performed in response to an indication of an event having occurred being provided by a user (for example, by the event sniffer 201 as described above).

It will be understood, as shown in FIGS. 2 and 5, that human assessment will be required for adjudication of some cases, for example those which have a low attributed probability score. It will therefore be incumbent on a healthcare provider or manager to ensure that these cases are monitored and adjudicated in a correct and timely manner. Accordingly, there is disclosed herein a method of monitoring clinical trial endpoint adjudication. The method comprises receiving a plurality of notifications of an adjudication decision from a clinical trial endpoint adjudication system, wherein the adjudication decisions comprise a probability score providing an indication of the likelihood of the event having occurred. The method then comprises ranking the notifications based on at least one of (i) the probability score and (ii) the severity of the event, and obtaining a dossier of data used in performing the adjudication (i.e. the Virtual Adjudication Dossier 1313). The method then comprises providing a list of adjudication decisions and the corresponding Virtual Adjudication Dossier 1313 (as shown in FIG. 11 and described above) of data to a user to review the correctness of the adjudication decision, wherein the order of the list is based on the ranking.

The method may further comprise obtaining a ranking of the importance of the features involved in classifying whether a healthcare event has occurred, and providing the ranking of the importance of the features to the user with the list of adjudication decisions and the corresponding dossier of data.

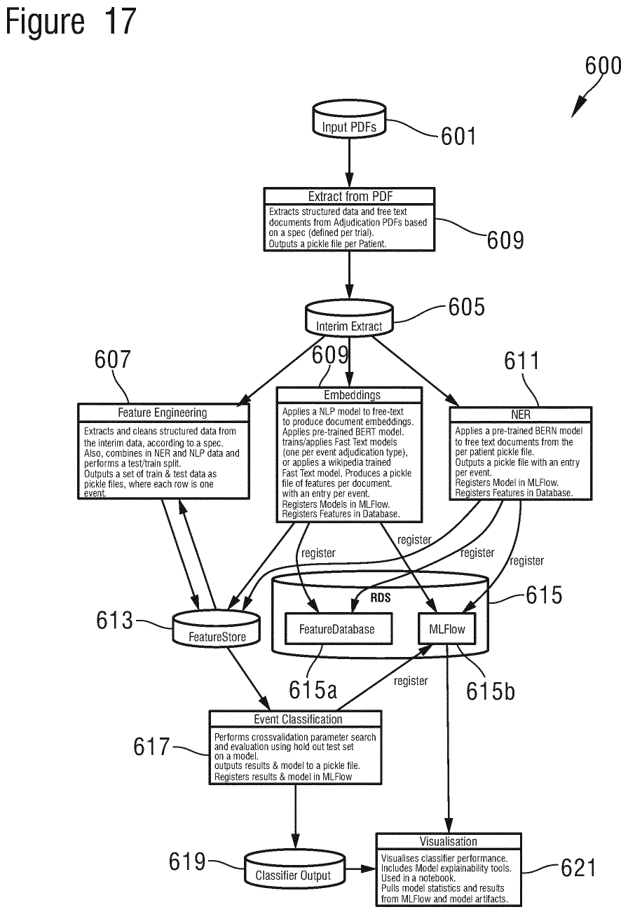

FIG. 17 shows at a high level the components (for example, for example modules that may be implemented in software and/or hardware) and flow of data between them, along with brief descriptions of the function of each component, operable for performing the method of FIG. 16 described above and for training a machine learning model for performing the method of FIG. 16.

In more detail, data (such as PDFs) are input at 601. At 603, structure data and unstructured data (free-text documents) are extracted, and in this example output as a pickle file as the system may be operating using the Python language. At 605 an interim extract is performed. 607 comprises feature engineering, where cleaned structured data is extracted from the interim data, according to a specification. In some example, this is combined with NLP data (from the embeddings at 609 described below) and NER (at 611 also described below). In some examples 697 also comprise performing a test/train split on the data for use in training the machine learning model(s).

At 609, embeddings are obtained. This involves applying a NLP model to free-text to produce document embeddings. This may involve applying a pre-trained bert model, fasttext models (one per event adjudication type), and/or a Wikipedia®-trained fasttext model. This may output a pickle file of features per document, with an entry per event. Models may be refistered in MLFlow 615b, and features may be registered in FeatureDatabase 615a. MLFlow 615b and FeatureDatabase 615a may both be part of remote data store (RDS) 615.

At 611 a pre-trained bern model may be applied to free text documents from the per patient pickle file. A pickle file with an entry per event may be output. The model may be registered in MLFlow 615b and features registered in FeatureDatabase 615a.

Each of 607, 609 and 611 may provides features for storing in FeatureStore 613. The FeatureStore 613 is used at 617 to perform event classification. This may perform a crossvalidation parameter search and evaluation using hold out test set on a model. Results and the model may be output to a pickle file and results and the model may be registered in MLFLow 615b.

At 621 visualisation is performed which visualises classifier performance (this will be described in more detail below with reference to FIGS. 20A, 20B, 21). It may pull model statistics and results from MLFlow 615b and model artefacts.

It will be understood that models used for the adjudication decision may need to be trained. Accordingly, with reference to FIG. 17 described above, a method of training a machine learning classification model for performing clinical trial endpoint adjudication is disclosed herein. It will be understood that the method of training may be performed by implementing the method and system of FIG. 17. The method comprises receiving data from a plurality of healthcare-related data sources, the data comprising adjudication dossiers from previous clinical trials and adjudication decisions relating to those adjudication dossiers. Each data source is analysed to determine whether data held by the data source comprises structured and/or unstructured data. In the event that the data comprises unstructured data, the method comprises applying a natural language processing model to the unstructured data to obtain embeddings relating to features in the unstructured data. In the event that the data comprises structured data, the method comprises extracting features from that data. The method then comprises providing an indication of the adjudication decision based on the data from the adjudication dossier and updating the machine learning classification model based on the adjudication decision and the data from the adjudication dossier. The updated machine learning classification model is then stored in a relational database.

After models have been trained and retrospectively validated for performance in the context of closed clinical trials, the best performing feature set and machine learning model may be selected for deployment in either a stand-alone manner or as an ensemble.

As described above, the “Automated Event Adjudication” module 205 may be used in combination with the Data harmonization and collection” module 203 and optionally the “event sniffer” module 201. FIG. 18 shows an example of how the “Automated Event Adjudication” module 205 may be used in combination with the “Data harmonization and collection” module 203. FIG. 18 shows the conceptual stages which may be applied to the process of automatically adjudicating an adverse event in a clinical trial.

As can be seen in FIG. 18, at 401 data is input to the Data harmonization and collection module 203. The data is split into an unstructured source document 403 or EDC (structured) data 405. For the unstructured source document 403 extraction 407 is performed to obtain extracted data (for example by using NLP to obtain embeddings and/or NER as described above). For structured data, the data is extracted 409 and extracted structured data 413 obtained. At 415 the structured and unstructured data are harmonized (for example, to ensure consistency of fields relevant to the clinical endpoints(s) concerned), to obtain harmonized data 417. At this stage a quality control check 419 is performed, for example using the QCBot 1305 as described above in reference to FIG. 111. The data is then passed to the “Automated Event Adjudication” module 205 where a model specific QC check 421 may be performed (for example, using the QCBot 1311). At 423 features are constructed to obtain a set of features 425 which are then used at 427 for classification. The result of the classification 427 is an adjudication output 429. In some cases (e.g. where there is relatively low probability of an event having occurred), manual adjudication 431 may then be performed.

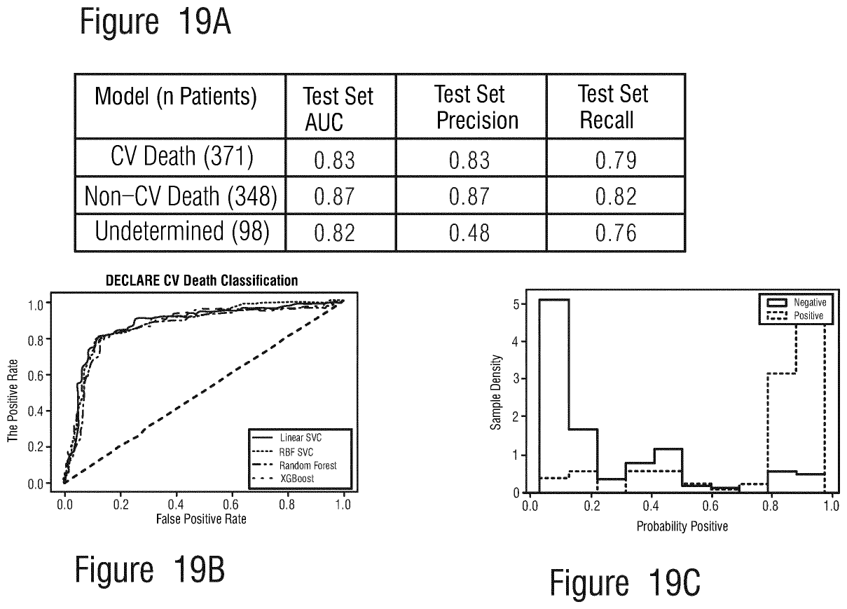

It will be understood that different machine learning algorithms may be trained and deployed depending upon the event (e.g. CV death) that they are designs to determine or adjudicate. For example, FIGS. 19A to 9C shows the results of 3 algorithms (CV Death, Non-CV Death and Undetermined) that were trained on 817 patients from the DECLARE clinical trial. It can be seen that whether a Linear SVC, RBF SVC, Random Forest or XGBoost model were applied to the data, the ROC curves are all relatively similar and close to the perfect classifier. FIG. 19A demonstrates that the Test set (i.e., hold-out set) AUC is >80% for each of the models. FIG. 19C shows the distribution of model predictive scores (X axis) by class (colour), and indicates that the model generally produces higher scores (closer to 1) for positive cases and generally produces lower scores (closer to 0) for negative cases. This separation tends to indicate an effective classifier that can distinguish between binary classes.

Machine learning models are extremely complex over the global feature space however they are much simpler locally. A particular test set may be perturbed to explain and learn a local linear approximation of the model's behaviour, as an explanation. This may reveal what effect a perturbation to the input has on the model prediction, and how each feature contributes to the model prediction. This may be termed Local Interpretable Model-agnostic Explanations or LIME.

FIGS. 20A and 20B show how the models or algorithms can be analysed to provide interpretability. For example, it can be seen how the models can be analysed to provide an indication of the top features with mutual information for structured data, and also for unstructured data. In FIG. 20A, the feature importance analysis for structured features in this model indicates that “CT Scan unknown” and “Neurological deficit on exam unknown” have the highest importance, meaning they are more useful than other structured features in predicting the target variable.

FIG. 20B enabled model interpretability by showing how the NLP algorithm (BioBERT, in this case) interprets and groups together similar terms based on their context in a body of text via transforming text into a vector of numbers. For example, in the lower right of the graphic, ‘coronary heart disease’ and ‘myocardial infarction’, both terms from the general domain of cardiovascular health, have been identified as similar by the BioBERT model and are thus close to each other relative to other terms on the chart. Visual inspection enabled by this chart confirms that the BioBERT model is working as expected.

SHapley Additive exPlanations or SHAP is a game theoretic approach to explain the output of any machine learning model. It connects optimal credit allocation with local explanations to understand:
How important is each feature to the overall model prediction?
What effect can a feature value be reasonably expected to have compared to its average?

SHAP values denote the impact of each feature on the model output. This can be represented in the chart shown, for example, in FIG. 21.

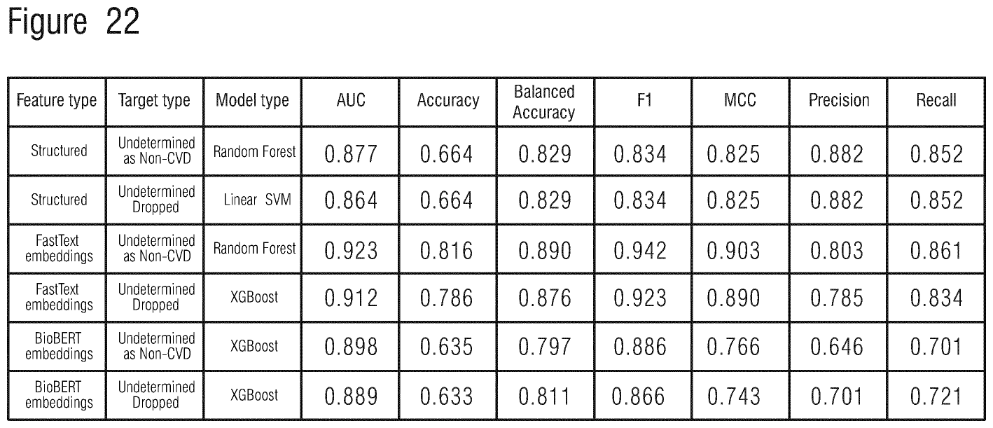

FIG. 22 shows machine learning model performance across a number of metrics (e.g., AUC-Area Under the Curve, Accuracy, Balanced Accuracy, F1, etc.) and model versions (first three columns). For example, the first row shows metrics (model performance) for a death event random forest algorithm trained on structured EDC data, with the undetermined class mapped to non-CV Death.

### Example 1

Accurate identification of clinical outcome events is critical to obtaining reliable results in cardiovascular outcomes trials (CVOTs). Current processes for event adjudication are expensive and hampered by delays. As part of a larger project to more reliably identify outcomes, we evaluated the use of machine learning to automate event adjudication using data from the SOCRATES trial (NCT01994720), a large randomized trial comparing ticagrelor and aspirin in reducing risk of major cardiovascular events after acute ischemic stroke or transient ischemic attack (TIA).

Machine learning algorithms were studied to determine whether they could replicate the outcome of the expert adjudication process for clinical events of ischemic stroke and TIA. Could classification models be trained on historical CVOT data and demonstrate performance comparable to human adjudicators?

Using data from the SOCRATES trial, multiple machine learning algorithms were tested using grid search and cross validation. Models tested included Support Vector Machines, Random Forest and XGBoost. Performance was assessed on a validation subset of the adjudication data not used for training or testing in model development. Metrics used to evaluate model performance were Receiver Operating Characteristic (ROC), Matthews Correlation Coefficient, Precision and Recall. The contribution of features, attributes of data used by the algorithm as it is trained to classify an event, that contributed to a classification were examined using both Mutual Information and Recursive Feature Elimination.

Classification models were trained on historical CVOT data using adjudicator consensus decision as the ground truth. Best performance was observed on models trained to classify ischemic stroke (ROC 0.95) and TIA (ROC 0.97). Top ranked features that contributed to classification of Ischemic Stroke or TIA corresponded to site investigator decision or variables used to define the event in the trial charter, such as duration of symptoms. Model performance was comparable across the different machine learning algorithms tested with XGBoost demonstrating the best ROC on the validation set for correctly classifying both stroke and TIA.

Results therefore indicate that machine learning may augment or even replace clinician adjudication in clinical trials, with potential to gain efficiencies, speed up clinical development, and retain reliability. Our current models demonstrate good performance at binary classification of ischemic stroke and TIA within a single CVOT with high consistency and accuracy between automated and clinician adjudication.

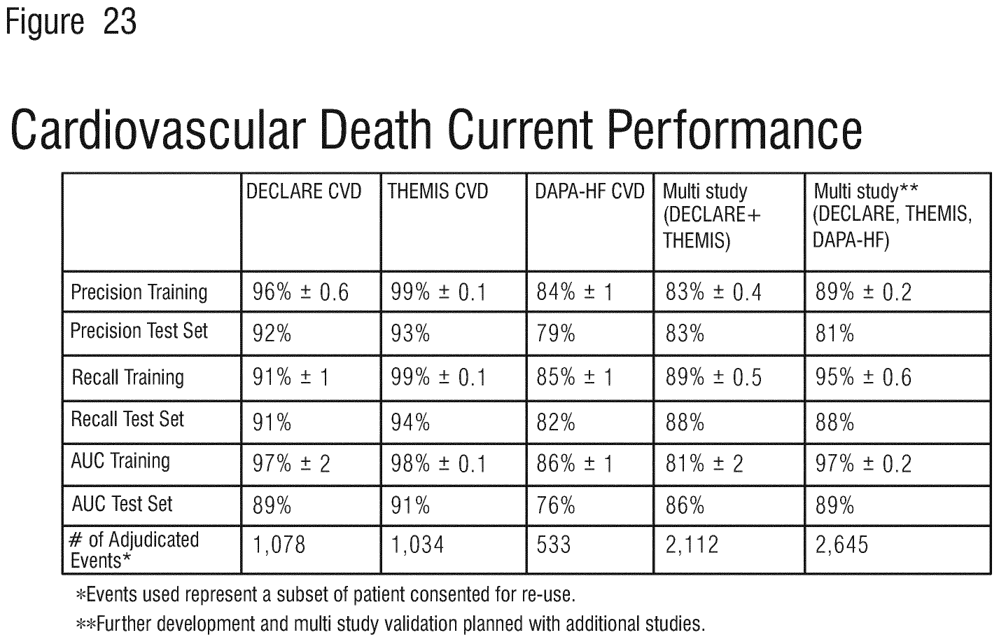

As further evidence of the efficacy of the models, FIG. 23 shows machine learning model performance when assessing cardiovascular death as an outcome across a number of metrics (e.g., AUC-Area Under the Curve, Accuracy, Balanced Accuracy, F1, etc.) performed on data from 3 clinical trials (DECLARE, THEM IS and DAPA-HF).

As noted above, the three modules (event sniffer module 201, data harmonization and collection module 203 and automated event adjudication module 205) as shown in FIG. 2 may make use of a machine learning model or ensemble of models. These models may comprise known models such as bert, biobert and fasttext, and/or adapted versions of these known models.

The machine learning model may comprise a neural network. The neural network may comprise at least one of a deep residual network, a highway network, a densely connected network and a capsule network.

For any such type of network, the network may comprise a plurality of different neurons, which are organised into different layers. Each neuron is configured to receive input data, process this input data and provide output data. Each neuron may be configured to perform a specific operation on its input, e.g. this may involve mathematically processing the input data. The input data for each neuron may comprise an output from a plurality of other preceding neurons. As part of a neuron's operation on input data, each stream of input data (e.g. one stream of input data for each preceding neuron which provides its output to the neuron) is assigned a weighting. That way, processing of input data by a neuron comprises applying weightings to the different streams of input data so that different items of input data will contribute more or less to the overall output of a neuron. Adjustments to the value of the inputs for a neuron, e.g. as a consequence of the input weightings changing, may result in a change to the value of the output for that neuron. The output data from each neuron may be sent to a plurality of subsequent neurons.

The neurons are organised in layers. Each layer comprises a plurality of neurons which operate on data provided to them from the output of neurons in preceding layers. Within each layer there may be a large number of different neurons, each of which applies a different weighting to its input data and performs a different operation on its input data. The input data for all of the neurons in a layer may be the same, and the output from the neurons will be passed to neurons in subsequent layers.

The exact routing between neurons in different layers forms a major difference between capsule networks and deep residual networks (including variants such as highway networks and densely connected networks).

For a residual network, layers may be organised into blocks, such that the network comprises a plurality of blocks, each of which comprises at least one layer. Fora residual network, output data from one layer of neurons may follow more than one different path. For conventional neural networks (e.g. convolutional neural networks), output data from one layer is passed into the next layer, and this continues until the end of the network so that each layer receives input from the layer immediately preceding it and provides output to the layer immediately after it. However, for a residual network, a different routing between layers may occur. For example, the output from one layer may be passed on to multiple different subsequent layers, and the input for one layer may be received from multiple different preceding layers.

In a residual network, layers of neurons may be organised into different blocks, wherein each block comprises at least one layer of neurons. Blocks may be arranged with layers stacked together so that the output of a preceding layer (or layers) feeds into the input of the next block of layers. The structure of the residual network may be such that the output from one block (or layer) is passed into both the block (or layer) immediately after it and at least one other later subsequent block (or layer). Shortcuts may be introduced into the neural network which pass data from one layer (or block) to another whilst bypassing other layers (or blocks) in between the two. This may enable more efficient training of the network, e.g. when dealing with very deep networks, as it may enable problems associated with degradation to be addressed when training the network (which is discussed in more detail below). The arrangement of a residual neural network may enable branches to occur such that the same input provided to one layer, or block of layers, is provided to at least one other layer, or block of layers (e.g. so that the other layer may operate on both the input data and the output data from the one layer, or block of layers). This arrangement may enable a deeper penetration into the network when using back propagation algorithms to train the network. For example, this is because during learning, layers, or blocks of layers, may be able to take as an input, the input of a previous layer/block and the output of the previous layer/block, and shortcuts may be used to provide deeper penetration when updating weightings for the network.

For a capsule network, layers may be nested inside of other layers to provide ‘capsules’. Different capsules may be adapted so that they are more proficient at performing different tasks than other capsules. A capsule network may provide dynamic routing between capsules so that for a given task, the task is allocated to the most competent capsule for processing that task. For example, a capsule network may avoid routing the output from every neuron in a layer to every neuron in the next layer. A lower level capsule is configured to send its input to a higher level (subsequent) capsule which is determined to be the most likely capsule to deal with that input. Capsules may predict the activity of higher layer capsules. For example, a capsule may output a vector, for which the orientation represents properties of an object in question. In response, each subsequent capsule may provide, as an output, a probability that the object that capsule is trained to identify is present in the input data. This information (e.g. the probabilities) can be fed back to the capsule, which can then dynamically determine routing weights, and forward the input data to the subsequent capsule most likely to be the relevant capsule for processing that data.

For either type of neural network, there may be included a plurality of different layers which have different functions. The neural network may include at least one convolutional layer configured to convolve input data across its height and width. The neural network may also have a plurality of filtering layers, each of which comprises a plurality of neurons configured to focus on and apply filters to different portions of the input data. Other layers may be included for processing the input data such as pooling layers (to introduce non-linearity) such as maximum pooling and global average pooling, Rectified Linear Units layer (ReLU) and loss layers, e.g. some of which may include regularization functions. The final block of layers may receive input from the last output layer (or more layers if there are branches present). The final block may comprise at least one fully connected layer.

The final output layer may comprise a classifier, such as a softmax, sigmoid or tan h classifier. Different classifiers may be suitable for different types of output; for example, a sigmoid classifier may be suitable where the output is a binary classifier. The output of the neural network may provide an indication of a probability.

Inputs may be fed into a set of 3D layers in the neural network. There are several features of this network which may be varied as training of the network proceeds. For each neuron, there may be a plurality of weightings, each of which is applied to a respective input stream for output data from neurons in preceding layers. These weightings are variables which can be modified to provide a change to the output of the neural network. These weightings may be modified in response to training so that they provide more accurate data. In response to having trained these weightings, the modified weightings are referred to as having been ‘learned’. Additionally, the size and connectivity of the layers may be dependent upon the typical input data for the network; although, these too may be a variable which may be modified and learned during training, including the reinforcement of connections.

To train the network, e.g. to learn values for the weightings, these weightings are assigned an initial value. These initial values may essentially be random; however, to improve training of the network, a suitable initialisation for the values may be applied such as a Xavier/Glorot initialisation. Such initialisations may inhibit situations from occurring in which initial random weightings are too great or too small, and the neural network can never properly be trained to overcome these initial prejudices. This type of initialisation may comprise assigning weightings using a distribution having a zero mean but a fixed variance.

Once the weightings have been assigned, training object data may be fed or input into the neural network. This may comprise operating the neural network on the results of previous clinical trial adjudication decisions, as referenced above when describing FIG. 6. Algorithms such as mini-batch gradient descent, RMSprop, Adam, Adadelta and Nesterov may be used during this process. This may enable an identification of how much each different point (neuron) or path (between neurons in subsequent layers) in the network is contributing to determining an incorrect score, thus enabling a determination of any weight adjustments that need to be made. The weightings may then be adjusted according to the error calculated. For example, to minimise or remove the contribution from neurons which contribute, or contribute the most, to an incorrect determination.

After an iteration of training the network, the weightings may be updated 750, and this process may be repeated a large number of times. To inhibit the likelihood of overtraining the network, training variables such as learning rate and momentum may be varied and/or controlled to be at a selected value. Additionally, regularisation techniques such as L2 or dropout may be used which reduce the likelihood of different layers becoming over-trained to be too specific for the training data, without being as generally applicable to other, similar data. Likewise, batch normalisation may be used to aid training and improve accuracy. In general, the weightings are adjusted so that the network would, if operated on the same training data again, produce the expected outcome. Although, the extent to which this is true will be dependent on training variables such as learning rate.

It is to be appreciated that increasing the depth of neural networks may cause problems when training, e.g. due to vanishing gradient problems, and it may also provide slower networks. However, the present disclosure may enable the provision of a network having increased depth and accuracy without sacrificing the ability to adequately train the network.

The depth of the network used may be selected to provide a balance between accuracy and the time taken to provide an output. Increasing the depth of the network may provide increased accuracy although it may also increase the time taken to provide an output. Use of a branched structure (as opposed to in a convolutional neural network) may enable sufficient training of the network to occur as depth of the network increases, which in turn provides for an increased accuracy of the network.

FIG. 24 is a block diagram of a computer system 2600 suitable for implementing one or more embodiments of the present disclosure. In various implementations, the computer system 2600 may include a user device such as a mobile cellular phone, personal computer (PC), laptop, wearable computing device, tablet etc. adapted for wireless communication. However, it will also be understood that in some examples embodiments of the disclosure may be implemented in the cloud on a remote server, and similar functionality as indicated in FIG. 24 may be provided by the remote server.

The computer system 2600 includes a bus 2612 or other communication mechanism for communicating information data, signals, and information between various components of the computer system 2600. The components include an input/output (I/O) component 2604 that processes a user (i.e., sender, recipient, service provider) action, such as selecting keys from a keypad/keyboard, selecting one or more buttons or links, etc., and sends a corresponding signal to the bus 2612. The I/O component 604 may also include an output component, such as a display 2602 and a cursor control 2608 (such as a keyboard, keypad, mouse, etc.). The display 2602 may be configured to present a login page for logging into a user account or a checkout page for purchasing an item from a merchant. An optional audio input/output component 2606 may also be included to allow a user to use voice for inputting information by converting audio signals. The audio I/O component 2606 may allow the user to hear audio. A transceiver or network interface 2620 transmits and receives signals between the computer system 2600 and other devices, such as another user device, a merchant server, or a service provider server via network 2622. In one embodiment, the transmission is wireless, although other transmission mediums and methods may also be suitable. A processor 2614, which can be a micro-controller, digital signal processor (DSP), or other processing component, processes these various signals, such as for display on the computer system 2600 or transmission to other devices via a communication link 2624. The processor 2614 may also control transmission of information, such as cookies or IP addresses, to other devices.

The components of the computer system 2600 also include a system memory component 2610 (e.g., RAM), a static storage component 2616 (e.g., ROM), and/or a disk drive 2618 (e.g., a solid-state drive, a hard drive). The computer system 600 performs specific operations by the processor 614 and other components by executing one or more sequences of instructions contained in the system memory component 2610.

Logic may be encoded in a computer readable medium, which may refer to any medium that participates in providing instructions to the processor 2614 for execution. Such a medium may take many forms, including but not limited to, non-volatile media, volatile media, and transmission media. In various implementations, non-volatile media includes optical or magnetic disks, volatile media includes dynamic memory, such as the system memory component 2610, and transmission media includes coaxial cables, copper wire, and fiber optics, including wires that comprise the bus 2612. In one embodiment, the logic is encoded in non-transitory computer readable medium. In one example, transmission media may take the form of acoustic or light waves, such as those generated during radio wave, optical, and infrared data communications.

Some common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer is adapted to read.

In various embodiments of the present disclosure, execution of instruction sequences to practice the present disclosure may be performed by the computer system 2600. In various other embodiments of the present disclosure, a plurality of computer systems 2600 coupled by the communication link 2624 to the network (e.g., such as a LAN, WLAN, PTSN, and/or various other wired or wireless networks, including telecommunications, mobile, and cellular phone networks) may perform instruction sequences to practice the present disclosure in coordination with one another.

Where applicable, various embodiments provided by the present disclosure may be implemented using hardware, software, or combinations of hardware and software. Also, where applicable, the various hardware components and/or software components set forth herein may be combined into composite components comprising software, hardware, and/or both without departing from the spirit of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software in accordance with the present disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The various features and steps described herein may be implemented as systems comprising one or more memories storing various information described herein and one or more processors coupled to the one or more memories and a network, wherein the one or more processors are operable to perform steps as described herein, as non-transitory machine-readable medium comprising a plurality of machine-readable instructions which, when executed by one or more processors, are adapted to cause the one or more processors to perform a method comprising steps described herein, and methods performed by one or more devices, such as a hardware processor, user device, server, and other devices described herein.

In the context of the present disclosure other examples and variations of the apparatus and methods described herein will be apparent to a person of skill in the art.

1. A computer-implemented method for performing clinical trial endpoint adjudication, the method comprising:
at a computing system, receiving data from a plurality of healthcare-related data sources;
in the event that the data comprises unstructured data, applying a natural language processing model to the unstructured data to obtain embeddings relating to features in the unstructured data;
in the event that the data comprises structured data, extracting features from that data;
applying a machine learning classification model to the embeddings from the unstructured data and the features extracted from the structured data to classify whether a healthcare event has occurred based on the embeddings and the features extracted from the structured data;
attributing a probability score as an attribute to the classification, wherein the probability score provides an indication of the likelihood of the event having occurred;
providing a notification to a user to review classifications where the probability score is less than a selected threshold.

2. The computer-implemented method of claim 1 wherein applying a natural language processing model comprises applying a plurality of natural language processing models, comprising a first specialised model trained on text available from the data sources, with a second general model.

3. The computer-implemented method of claim 1 or 2 further comprising, in the event that the data comprises unstructured data, applying a named-entity recognition model to the unstructured data to obtain formal event characteristics from the unstructured data; and
applying the machine learning classification model to the formal event characteristics obtained via the named-entity recognition model.

4. The computer-implemented method of any of the previous claims attributing a confidence score as an attribute to the data based on at least one of (i) the data source, and (ii) a determined confidence based on an optical character recognition process applied to the unstructured data;
and using the confidence score as a weighting by the machine learning classification model.

5. The computer-implemented method of claim 4 further comprising excluding data having a confidence score below a selected threshold.

6. The computer-implemented method of any of the previous claims, wherein extracting features from the data and applying a natural language processing model to the unstructured data to obtain embeddings relating to features in the unstructured data comprises obtaining a predefined set of features for use in the clinical endpoint adjudication and mapping the extracted features and/or embeddings to the predefined set of features, and discarding features and/or embeddings that do not relate to the predefined set of features.

7. The computer-implemented method of any of the previous claims wherein the machine learning classification model provides a ranking of the importance of the features involved in classifying whether a healthcare event has occurred.

8. The computer-implemented method of claim 7 wherein providing a ranking of the importance of features comprises determining the SHAP value for each feature.

9. The computer-implemented method of claim 7 or 8 wherein providing a ranking of the importance of features comprises applying a local surrogate model to the machine learning classification model to determine the relative contribution of each feature to the classification.

10. The computer-implemented method of any of the previous claims wherein the method is performed when the amount of data available exceeds a selected threshold.

11. The computer-implemented method of any of the previous claims wherein the method is performed in response to an indication of an event having occurred being provided by a user.

12. A method of training a machine learning classification model for performing clinical trial endpoint adjudication, the method comprising:
at a computing system, receiving data from a plurality of healthcare-related data sources, the data comprising adjudication dossiers from previous clinical trials and adjudication decisions relating to those adjudication dossiers;
analysing each data source to determine whether data held by the data source comprises structured and/or unstructured data;
in the event that the data comprises unstructured data, applying a natural language processing model to the unstructured data to obtain embeddings relating to features in the unstructured data;
in the event that the data comprises structured data, extracting features from that data;
providing an indication of the adjudication decision based on the data from the adjudication dossier;
updating the machine learning classification model based on the adjudication decision and the data from the adjudication dossier;
storing the updated machine learning classification model in a relational database.

13. A method of monitoring clinical trial endpoint adjudication, the method comprising:
at a computing system, receiving a plurality of notifications of an adjudication decision from a clinical trial endpoint adjudication system, wherein the adjudication decision comprises a probability score providing an indication of the likelihood of the event having occurred;
ranking the notifications based on at least one of (i) the probability score and (ii) the severity of the event;
obtaining a dossier of data used in performing the adjudication;
providing a list of adjudication decisions and the corresponding dossier of data to a user to review the correctness of the adjudication decision, wherein the order of the list is based on the ranking.

14. The method of claim 13 further comprising:
obtaining a ranking of the importance of the features involved in classifying whether a healthcare event has occurred; and
providing the ranking of the importance of the features to the user with the list of adjudication decisions and the corresponding dossier of data.

15. A computer-implemented method of harmonising and collating data from a plurality of healthcare-related sources for clinical trial endpoint adjudication, the method comprising:
at a computing system, analysing each data source to determine whether data held by the data source comprises structured and/or unstructured data;
in the event that the data comprises unstructured data, performing optical character recognition on a region or regions of the data not already in a machine-readable format;
attributing a confidence score as an attribute to the data based on at least one of (i) the data source, and (ii) a determined confidence based on the optical character recognition process;
performing a feature analysis on the data to extract features from the data;
mapping the extracted features to a predefined set of features;
publishing the mapped extracted features in a json format for use by a machine learning model in performing the clinical trial endpoint adjudication, wherein the confidence score is an attribute of the feature.

16. The method of claim 15 further comprising publishing the mapped extracted features in a json format when the confidence score is above a selected confidence threshold.

17. The method of claim 15 or 16 further comprising:
obtaining a set of features needed for a clinical trial endpoint adjudication, wherein the set of features needed is based on the endpoint;
comparing the features obtained from the plurality of data sources with the set of features needed for a clinical trial endpoint adjudication to determine whether any features are missing or incomplete;
in the event that it is determined that any features are missing or incomplete, providing a notification to a user that features are missing, the notification providing an indication of the missing or incomplete features.

18. The method of any of the previous claims further comprising performing named-entity recognition on the data prior to performing a feature analysis on the data and selecting formal event characteristics that are relevant to the predefined set of features.

19. The method of any of the previous claims further comprising obtaining a set of features that should be provided by a data source, and determining whether any features are missing for that data source, and in the event that features are missing for that data source, providing a notification to a user that features are missing.

20. The method of any of the previous claims wherein performing a feature analysis on the data to extract features from the data further comprises checking for and removing any duplicated, inconsistent or inapplicable features.

21. A monitoring system for determining whether a healthcare event has occurred for a participant in a clinical trial, the system comprising:
a communications interface configured to receive data signals relating to a plurality of participants from a plurality of sources, wherein the data signals each comprise information indicative of a parameter associated with a participant; and
a processor;
wherein, for each participant, the processor is configured to process each received data signal and apply a first weighting to each data signal based on the source of the data signal;
wherein the processor is configured to make a determination of the probability of a healthcare event occurring based on at least one of: (i) the data signal indicating that a parameter associated with a patient exceeds a selected threshold for that participant, and (ii) the first weighting exceeding a selected trigger threshold.

22. The system of claim 11 wherein the processor is configured to make a determination that a healthcare event has occurred based on the determined probability exceeding a selected threshold, and in the event that the processor determines that a healthcare event has occurred, the processor is configured to provide a notification to a user of the monitoring system, and wherein the monitoring system is configured to rank the notifications based on the determined probability of the healthcare event occurring.

23. The system of claim 21 or 22 wherein the processor is configured to make a determination of the type of healthcare event based on the source of the data signal and the indication of the parameter associated with the participant.

24. The system of claim 23 wherein the monitoring system is configured to rank the notifications based on the determined type of healthcare event.

25. The system of claim 22, 23 or 24 wherein the monitoring system is configured to rank the notifications based on the known health of the participants.

26. The system of any of the previous claims wherein the processor is configured to make a determination of the probability of a healthcare event occurring based on a plurality of data signals with at least one data signal indicating at least one of (i) that a parameter associated with a patient exceeds a selected threshold and that (ii) a weighting of that data signal exceeds a selected threshold.

27. The system of any of the previous claims wherein when the processor makes a determination of the probability of a healthcare event occurring based on a received data signal indicating that a parameter has exceeded a selected threshold, the processor reviews information indicative of previous values of that parameter associated with the participant in a selected time interval preceding the determination.

28. The system of any of the previous claims, wherein if it is determined that the probability of an event having occurred exceeds a selected threshold, then the processor is configured to make a determination as to whether more information is needed from the patient, and in the event that more information is needed, then a notification is provided to the health care provider/system administrator to contact the patient.

29. The system of any of the previous claims wherein the processor is also configured to make a determination of the reliability of the data signal, and to apply a second weighting based on the reliability of the data signal, and wherein the processor is configured to make a determination of the probability of a healthcare event occurring based on the data signal indicating that a parameter associated with a patient exceeds a selected threshold and the first and second weightings.

30. The system of any of the previous claims wherein the processor is configured to make a determination of the probability of a healthcare event occurring for a participant also based on any previous determined probabilities of an event occurring for that participant.

31. A method for determining whether a healthcare event has occurred for a participant in a clinical trial, the method comprising:
at a computing system, receiving data signals relating to a plurality of participants from a plurality of sources, wherein the data signals each comprise information indicative of a parameter associated with a participant; and
for each participant, processing each received data signal and applying a first weighting to each data signal based on the source of the data signal;
determining the probability of a healthcare event occurring based on at least one of: (i) the data signal indicating that a parameter associated with a patient exceeds a selected threshold for that participant, and (ii) the first weighting exceeding a selected trigger threshold.

32. The method of claim 31 comprising determining that a healthcare event has occurred based on the determined probability exceeding a selected threshold, and in the event that it is determined that a healthcare event has occurred, providing a notification to a user, wherein the notification is ranked based on the determined probability of the healthcare event occurring.

33. The method of claim 31 comprising determining the type of healthcare event based on the source of the data signal and the indication of the parameter associated with the participant.

34. The method of claim 33 comprising ranking the notifications based on the determined type of healthcare event.

35. The method of claim 32 comprising ranking the notifications based on the known health of the participants.

36. The method of claim 31 comprising making a determination of the probability of a healthcare event occurring based on a plurality of data signals with at least one data signal indicating at least one of (i) that a parameter associated with a patient exceeds a selected threshold and that (ii) a weighting of that data signal exceeds a selected threshold.

37. The method of claim 31 comprising making a determination of the probability of a healthcare event occurring based on a received data signal indicating that a parameter has exceeded a selected threshold, wherein the determination is based on information indicative of previous values of that parameter associated with the participant in a selected time interval preceding the determination.

38. The method of claim 31 wherein if it is determined that the probability of an event having occurred exceeds a selected threshold, then the processor is configured to make a determination as to whether more information is needed from the patient, and in the event that more information is needed, then a notification is provided to the health care provider/system administrator to contact the patient.

39. The method of claim 31 comprising making a determination of the reliability of the data signal, and applying a second weighting based on the reliability of the data signal, and wherein the method comprises making a determination of the probability of a healthcare event occurring based on the data signal indicating that a parameter associated with a patient exceeds a selected threshold and the first and second weightings.

40. The method of any of the previous claims comprising making a determination of the probability of a healthcare event occurring for a participant also based on any previous determined probabilities of an event occurring for that participant.

41. A monitoring system for determining whether a healthcare event has occurred for a participant in a clinical trial, the system comprising:
a communications interface configured to receive data signals relating to a plurality of participants from a plurality of sources, wherein the data signals each comprise information indicative of a location associated with a participant; and
a processor;
wherein, for each participant, the processor is configured to process each received data signal; and
wherein the processor is configured to make a determination of the probability of a healthcare event occurring for a participant based on (i) the proximity of the participant to a known healthcare centre and (ii) the duration of the participant's proximity to the known healthcare centre; and
in the event that the processor determines that the probability of a healthcare event occurring exceeds a selected threshold, the processor is configured to send a notification to the participant requesting confirmation from the participant that a healthcare event has occurred.

42. A method for determining whether a healthcare event has occurred for a participant in a clinical trial, the method comprising:
at a computing system, receiving data signals relating to a plurality of participants from a plurality of sources, wherein the data signals each comprise information indicative of a location associated with a participant; and
processing, for each participant, each received data signal to make a determination of the probability of a healthcare event occurring for a participant based on (i) the proximity of the participant to a known healthcare centre and (ii) the duration of the participant's proximity to the known healthcare centre; and
in the event that it is determined that the probability of a healthcare event occurring exceeds a selected threshold, sending a notification to the participant requesting confirmation from the participant that a healthcare event has occurred.

43. A computer readable non-transitory storage medium comprising a program for a computer configured to cause a processor to perform the method of claim 1.

44. A computer readable non-transitory storage medium comprising a program for a computer configured to cause a processor to perform the method of claim 12.

45. A computer readable non-transitory storage medium comprising a program for a computer configured to cause a processor to perform the method of claim 13.

46. A computer readable non-transitory storage medium comprising a program for a computer configured to cause a processor to perform the method of claim 15.

47. A computer readable non-transitory storage medium comprising a program for a computer configured to cause a processor to perform the method of claim 31.

48. A computer readable non-transitory storage medium comprising a program for a computer configured to cause a processor to perform the method of claim 42.
